(19) United States
(12) Patent Application Publication
(10) Pub. No.: US 2006/0122152 A1
(43) Pub. Date: Jun. 8, 2006

(54) Heparin for the treatment of ocular pathologies

(76) Inventor: Gholam A. Peyman, New Orleans, LA (US)

Correspondence Address: FROMMER LAWRENCE & HAUG, 745 FIFTH AVENUE- 10TH FL., NEW YORK, NY, 10151, US

(21) Appl. No.: 11/067,469

(22) Filed: Feb. 25, 2005

(30) Foreign Application Priority Data
Dec. 3, 2004 (AU) 2004906932
Dec. 3, 2004 (AU) 2004906934

(51) Int. Cl. A61K 31/727; A61K 31/65

(52) U.S. Cl. 514/56; 514/154

(57) ABSTRACT

Formulations and methods useful to treat ocular neovascularization (new blood vessel growth in the cornea, retina, conjunctiva, and/or choroid) are illustrated. According to the invention there is provided a formulation suitable for the treatment of ocular neovascularization that may comprise heparin in a concentration and dose suitable for treating ocular neovascularization, characterized in that said compound is at a substantially neutral pH in a pharmaceutically acceptable form suitable for delivery to the eye. Use of drugs like steroids in the treatment of such ocular neovascularization ailments can increase intraocular pressure (glaucoma).

### INCORPORATION BY REFERENCE

This application claims benefit of U.S. patent application Ser. No. 10/787,580 filed 26 Feb. 2004, U.S. patent application Ser. No. 10/828,982 filed 21 Apr. 2004, U.S. patent application Ser. No. 10/935,850 filed 8 Sep. 2004, U.S. patent application Ser. No. 10/936,303 filed 8 Sep. 2004, Australian Provisional Patent Application No. 2004906932 filed 3 Dec. 2004 and Australian Provisional Patent Application No. 2004906934 filed 3 Dec. 2004.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

Illustrated herein are formulations for the treatment of ocular neovascularization as well as treatment regimens that limit, reduce, slow the rate of, or prevent ocular neovascularization, and/or that cause regression of existing new blood vessels, in a patient with an ocular pathology.

### BACKGROUND OF THE INVENTION

Ocular neovascularization is the pathologic in-growth of blood vessels in the cornea, retina, or choroid. Blood vessel growth or formation can be due to diverse events and may lead to sight threatening conditions and even blindness due to bleeding and subsequent scarring, fibrosis, etc. Causes of blood vessel growth or formation include hypoxia (e.g., in diabetes), inflammatory responses (e.g., keratitis due to autoimmune disease), microbial infection (e.g., keratitis, blepharitis), physical insult (e.g., improper use of contact lenses), chemical insult (e.g., toxins), pharmacologic agents, or other factors (e.g., graft rejection). More specifically, an inflammatory response may follow corneal transplant. Ocular microbial infections include but are not limited to trachoma, viral interstitial keratitis, and keratoconjunctivitis. Physical insult (such as corneal insult) may be due to contact with acidic or alkaline solutions, trauma, improper hygiene and/or compliance with contact lens use, such as extended wear lenses, or chemical agents such as silver nitrate. Other factors leading to ocular neovascularization include mechanical irritation of the limbal sulcus, corneal hypoxia, epithelial cell erosion or hypertrophy. In dry eye disease (conjunctiva sicca), the dehydrated conditions cause sloughing off of the epithelium, resulting in new vessel formation.

One form of ocular neovascularization that is a major public health problem is neovascular disease of the cornea, which is a major cause of ocular morbidity. In the USA alone, corneal neovascularization (CoNV) is estimated to occur in 1.4 million patients (4% of the U.S. population) each year. CoNV may occur in a wide range of diseases affecting the cornea. For example, it may result from inflammatory conditions (such as chemical burns), immunologically mediated conditions (such as herpes simplex keratitis), allograft reactions, or extended wear contact lenses. These insults can lead to invasion of capillaries from the limbal plexus, resulting in CoNV which may lead to decreased visual acuity secondary to stromal edema, lipidic deposits, causal keratitis, and scarring.

The pathogenesis of CoNV has not yet been fully clarified in terms of identification and significance of angiogenic and anti-angiogenic factors. What is known is that corneal avascularity requires a balance between angiogenic and anti-angiogenic molecules. If this homeostasis is disrupted, it may result in neovascularization. More particularly, CoNV occurs when there is up-regulation of angiogenic factors or down regulation of anti-angiogenic molecules. Several angiogenic and anti-angiogenic molecules have been isolated from the cornea.

Numerous substances accelerate new vessel formation such as various growth factors (fibroblast growth factor, transforming growth factor, tumour necrosis factor, etc.), prostaglandins and interleukins. Various compounds have been identified as inhibitors in experimental and clinical CoNV including steroids, nonsteroidal anti-inflammatory drugs, cyclosporin A, methotrexate, FK506, thalidomide and other anti-angiogenic factors.

Methods of treating ocular neovascularization have met with limited success. Although there is no clear consensus, methods include treatment of the underlying condition, if possible; topical corticosteroid application for gross and active neovascularization; diathermy of large feeding vessels and corneal laser photocoagulation for treatment of superficial neovascularization of the cornea with infiltration of granulation tissue (pannus); and even timbal grafting for severe chemical injuries and limbal epithelium loss.

Topical corticosteroids have been the mainstay of prevention and treatment for CoNV, but they are not always effective and sometimes may be associated with serious complications such as cataract, ocular hypertension, glaucoma, and infections. Recognition of the potential side effects of corticosteroids in their use for angiogenesis has led to a search for other natural or synthetic angiogenesis inhibitors. Although corticosteroids have been known for a long time to be useful agents in prevention of CoNV in various clinical and experimental circumstances, there has not been enough research related with usage in combination with other drugs.

Other methods and formulations which reduce or prevent ocular neovascularization, and which may treat an ocular pathology, are desirable.

Citation or identification of any document in this application is not an admission that such document is available as prior art to the present invention.

### SUMMARY OF THE INVENTION

The invention relates to formulations and methods useful to treat ocular neovascularization (new blood vessel growth in the cornea, retina, conjunctiva, and/or choroid). According to the invention there is provided a formulation suitable for the treatment of ocular neovascularization comprising heparin in a concentration and dose suitable for treating ocular neovascularization, characterized in that said compound is in a pharmaceutically acceptable form suitable for delivery to the eye. Use of drugs like steroids in the treatment of such ocular neovascularization ailments can increase intraocular pressure (glaucoma). Accordingly a formulation according to the present invention is at least beneficial for patients with glaucoma or at risk for glaucoma, and for patients after glaucoma filtering surgery.

Desirably, there is provided a formulation suitable for the treatment of ocular neovascularization which may comprise heparin in a concentration and dose suitable for treating ocular neovascularization, characterized in that said compound is at a substantially neutral pH in a pharmaceutically acceptable form suitable for delivery to the eye.

According to the invention pharmaceutically acceptable formulations are desirably prepared for topical ocular application, ocular injection, or ocular implantation, and may be contained in liposomes or slow release capsules.

Preferably the formulation may comprise low molecular weight heparin in a concentration and dose suitable for treating ocular neovascularization (such as about 0.01 pg/ml to about 100 mg/ml low molecular weight heparin), characterized in that said compound is at a substantially neutral pH in a pharmaceutically acceptable form suitable for delivery to the eye.

In a more preferable form of the invention there is provided a pharmaceutically acceptable formulation suitable for treating ocular neovascularization that may comprise: (a) heparin (such as a low molecular weight heparin) in a concentration from about 0.01 pg/ml to about 100 mg/ml, characterized in that said compound is at a substantially neutral pH in a pharmaceutically acceptable form suitable for delivery to the eye; and (b) at least another compound in a concentration and dose sufficient to reduce ocular neovascularization selected from the group consisting of: a tetracycline, a steroid, an antimicrobial, an anti-prostaglandin, and/or a metalloproteinase inhibitor. More preferably, the formulation may include a plurality of compounds in a concentration and dose suitable for reducing ocular neovascularization selected from the group consisting of: a tetracycline, a steroid, an antimicrobial, an anti-prostaglandin, and/or a metalloproteinase inhibitor.

In a form of this embodiment, the invention relates to a formulation that may comprise: a low molecular weight heparin in a concentration from about 0.01 pg/ml to about 100 mg/ml, characterized in that said compound is at a substantially neutral pH in a pharmaceutically acceptable form suitable for delivery to the eye and a tetracycline derivative (such as, but not limited to, doxycycline, demeclocycline, minocycline, oxytetracycline, lymecycline, or a chemically modified tetracycline) at a concentration from about 0.01 pg/ml to about 30 mg/ml.

In a second form of this embodiment, the invention relates to a formulation that may comprise: a low molecular weight heparin in a concentration from about 0.01 pg/ml to about 100 mg/ml, characterized in that said compound is at a substantially neutral pH in a pharmaceutically acceptable form suitable for delivery to the eye and a steroid in a concentration and dose sufficient to reduce ocular neovascularization.

In a third form of this embodiment the invention relates to a formulation that may comprise: a low molecular weight heparin in a concentration from about 0.01 pg/ml to about 100 mg/ml, characterized in that said compound is at a substantially neutral pH in a pharmaceutically acceptable form suitable for delivery to the eye and an anti-prostaglandin in a concentration and dose sufficient to reduce ocular neovascularization.

For example, the formulation might comprise an actual concentration of 0.015% flurbiprofen.

In a fourth form of this embodiment the invention relates to a formulation that may comprise: a low molecular weight heparin in a concentration from about 0.01 pg/ml to about 100 mg/ml, characterized in that said compound is at a substantially neutral pH in a pharmaceutically acceptable form suitable for delivery to the eye and a antimicrobial in a concentration and dose sufficient to reduce ocular neovascularization.

In a fifth form of this embodiment the invention relates to a formulation that may comprise: a low molecular weight heparin in a concentration from about 00.01 pg/ml to about 100 mg/ml, characterized in that said compound is at a substantially neutral pH in a pharmaceutically acceptable form suitable for delivery to the eye and an inhibitor of a metalloproteinase in a concentration and dose sufficient to reduce ocular neovascularization.

In a sixth form of this embodiment the invention relates to a formulation that may comprise: a low molecular weight heparin in a concentration from about 0.01 pg/ml to about 100 mg/ml, characterized in that said compound is at a substantially neutral pH in a pharmaceutically acceptable form suitable for delivery to the eye and a steroid in a concentration and dose sufficient to reduce ocular neovascularization and doxycycline in a concentration and dose sufficient to reduce ocular neovascularization.

In another form the invention relates to a method for reducing ocular neovascularization, wherein the method may comprise the steps of administering to the eye of a patient a low molecular weight heparin in a concentration from about 0.01 pg/ml to about 100 mg/ml, characterized in that said compound is at a substantially neutral pH in a pharmaceutically acceptable form suitable for delivery to the eye. Administration may be by topical, subconjunctival, and intraocular routes or ocular implants. The method may reduce neovascularization in the anterior and/or posterior portions of the eye, or in the cornea, retina, choroid, etc.

Other objects, features, and advantages of the instant invention, in its details as seen from the above, and from the following description when considered in light of the appended claims.

These and other embodiments are disclosed or are obvious from and encompassed by, the following Detailed Description.

### BRIEF DESCRIPTION OF THE DRAWINGS

Comprehension of the invention is facilitated by reading the following detailed description, in conjunction with the annexed drawings.

The following detailed description, given by way of example, but not intended to limit the invention solely to the specific embodiments described, may best be understood in conjunction with the accompanying drawings, in which:

FIG. 1 is a photograph of a rat eye 7 days after administration of saline control.

FIG. 2 is a photograph of a rat eye 3 weeks after administration of a formulation containing 20 mg/ml doxycycline and 4 mg/ml triamcinolone acetonide.

FIG. 3A is a photograph of a rat eye to which flurbiprofen and low molecular weight heparin were administered.

FIG. 3B is a photograph of a rat eye to which flurbiprofen and doxycycline were administered.

FIG. 3C is a photograph of a rat eye to which doxycycline and low molecular weight heparin were administered.

FIG. 3D is a photograph of a rat eye to which a balanced salt solution was administered.

FIG. 4 is a graph showing the effect of various agents, on percentage of the cornea occupied by blood vessels.

FIG. 5A is a photograph of a histological preparation of a rat eye to which flurbiprofen and doxycycline were administered.

FIG. 5B is a photograph of a histological preparation of a rat eye to which a balanced salt solution was administered.

FIG. 6 is a bar chart demonstrating the percentage of cornea occupied by blood vessels in each group (LMWH: low molecular weight heparin, ASC: ascomycin, Flur: flurbiprofen, DOX: doxycycline, and TA: triamcinolone). Lines under the x-axis connect groups that are not significantly different from each other (p>0.5).

FIG. 7A is a slit lamp photograph of the cornea seven days after induction of corneal burn in control eyes receiving normal saline.

FIG. 7B is a digitally enhanced version of FIG. 7A, accentuating the blood vessels.

FIG. 8A is a digitally enhanced slit lamp photograph of the cornea seven days after induction of corneal burn in eyes treated with flurbiprofen (neovascularization is quite prominent in this group).

FIG. 8B is a digitally enhanced slit lamp photograph of the cornea seven days after induction of corneal burn in eyes treated with doxycycline (neovascularization is less prominent than in control group).

FIG. 8C is a digitally enhanced slit lamp photograph of the cornea seven days after induction of corneal burn in eyes treated with triamcinolone acetonide (arrows circumscribe the relatively small neovascular area).

FIG. 9A is a photograph of a histopathology preparation of the corneal burn in a control eye treated with normal saline, showing corneal scar (large arrow) and new vessels (small arrows) in the corneal stroma. H&E 100X.

FIG. 9B is a photograph of a histopathology preparation of the corneal burn in an eye treated with triamcinolone acetonide (double arrows point to avascular stroma). Note extensive neovascularization of the corneal stroma in FIG. 13A compared to FIG. 13B. H&E 100X.

FIG. 10A is a slit lamp photograph of the cornea 7 days after induction of corneal burn in a control animal administrated saline (advanced corneal neovascularization extending from the periphery to corneal burn).

FIG. 10B is a digitally enhanced version of FIG. 10A, accentuating the blood vessels.

FIG. 10C is a digitally enhanced slit lamp photograph of the cornea 7 days after induction of corneal burn in an animal administered triamcinolone acetonide and low molecular weight heparin group (corneal neovascularization is seen at the periphery).

FIG. 10D is a digitally enhanced slit lamp photograph of the cornea 7 days after induction of corneal burn in an animal administered triamcinolone acetonide and doxycycline group (no corneal neovascularization is seen, the eye appears quiet).

FIG. 11 is a bar graph showing the means of percent area of corneal neovascularization in rats (TA: triamcinolone acetonide, LMWH: Low molecular weight heparin, Dx: Doxycycline).

FIG. 12A is a photograph of a histological preparation of a cornea after chemical burn treated with normal saline drops; note new vessels (long arrows) and inflammatory cells (short arrows) throughout the entire corneal stroma.

FIG. 12B is a photograph of a histological preparation of a cornea after chemical burn treated with triamcinolone acetonide and low molecular weight heparin; demonstrating some inflammatory cells (short arrows) near the corneal burn; note lack of neovascularization in the stroma.

FIG. 12C is a photograph of a histological preparation of a cornea after chemical burn treated with triamcinolone acetonide and doxycycline; note normal corneal structure without inflammatory cells and neovascularization: arrow head indicates edge of the cornea burn.

### DETAILED DESCRIPTION

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

The file of this patent contains at least one drawing executed in color. Copies of this patent with color drawing(s) will be provided by the Patent and Trademark Office upon request and payment of the necessary fee.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the description of the invention and apply throughout. Unless otherwise defined, all other scientific and technical terms used herein have the same meaning as commonly understood to one of ordinary skill in the art to which the invention belongs.

Illustrated herein are formulations for the treatment of ocular neovascularization as well as treatment regimens that limit, reduce, slow the rate of, or prevent ocular neovascularization, and/or that cause regression of existing or new blood vessels, generally referred to as reduced neovascularization, although the term encompasses any degree of inhibition by any method and also encompasses any degree of regression of existing vessels.

Ocular neovascularizations can be superficial or deep and may lead to loss of optical transparency through stromal hemorrhage, scarring, lipid deposition, etc. Neovascularization may occur in any area of the eye, such as the cornea, retina, conjunctiva, or choroid. The presence of new vessels may result in an increased intraocular pressure, termed neovascular glaucoma or ocular hypertension. The new vessels may lead to hemorrhage and fibrosis, and result in structural damage to the eye with subsequent decreased visual acuity. For example, corneal burns result in the formation of new vessels that can decrease vision as they infiltrate and penetrate the cornea. In corneal transplants, new blood vessels from the limbus penetrate the cornea and may result in rejection of the engrafted tissues. Thus, control or prevention of new vessels to any extent is desirable, although greater inhibition is more desirable and total inhibition of new vessels is most desirable.

As used herein the phrase “ocular neovascularization” refers to any ocular disorder or pathological condition of the eye, i.e. ocular disease, which is caused by vessel growth or proliferation as a component to the disease state. Such ocular diseases can include, inter alia, but are not limited to: ocular neovascularization; retinal diseases (such as diabetic retinopathy, sickle cell retinopathy, retinopathy of prematurity, macular degeneration (eg early onset macular degeneration, neovascular macular degeneration, age-related macular degeneration)); iritis; rubeosis iritis; inflammatory diseases; anterior and posterior uveitis including chronic uveitis; neoplasms (retinoblastoma, pseudoglioma); Fuchs' heterochromic iridocyclitis; neovascular glaucoma; corneal neovascularization (inflammatory, transplantation); ischemic retinopathies; sequelae vascular diseases (retinal ischemia, choroidal vascular insufficiency, choroidal thrombosis, carotid artery ischemia); choroidal neovascularization; pterygium; neovascularization of the optic nerve; neovascularization due to penetration of the eye or contusive ocular injury and exudative retinopathies like myopic retinopathies, cystoid macular edema arising from various aetiologies, exudative macular degeneration, diabetic macular edema, central vein occlusion, branch vein occlusion; retinitis of prematurity, cyclitis, sickle cell retinopathy and macular edema arising from laser treatment of the retina or as a post-operative treatment, e.g. after corneal transplant or ocular surgery including corneal surgery (e.g., LASIK® surgery, photorefractive keratectomy (PRK), or other corneal procedures. The inventive methods and formulations may desirably inhibit ocular neovascularization that occurs from any event, for example, due to ocular disease, hypoxia, trauma, physical or chemical insult, etc.

In various embodiments, doses and formulations of the inventive formulation are administered to a patient in addition to, or as treatments for, an ocular neovascularization pathology.

According to the invention there is provided a formulation suitable for the treatment of ocular neovascularization comprising heparin in a concentration and dose suitable for treating ocular neovascularization, characterized in that said compound is in a pharmaceutically acceptable form suitable for delivery to the eye. According to the invention the formulations are preferably in pharmaceutically acceptable formulations for topical ocular application, ocular injection, or ocular implantation, and may be contained in liposomes or slow release capsules.

It will be appreciated that the heparin employed in the invention need only be in a form where it can be administered to or applied to the eye or its surrounding tissue. Thus, the heparin may be prepared in an acidic or basic environment and or may even be provided in a final form suitable for administration in this form. Preferably however the heparin is prepared in a form suitable for administrations to the ocular environment. More preferably it is prepared in a manner which results in the final formulation having some physiologically compatibility with the eye. For example, if the formulation is to be injected into the eye the formulation should be physiologically suitable for ocular insertion. Likewise if the formulation is prepared for topical administration then it may be in a form that is not necessarily physiologically compatible with the ocular environment, but by the time the compound(s) reaches its site of action is so compatible.

Desirably there is provided a formulation suitable for the treatment of ocular neovascularization comprising heparin in a concentration and dose suitable for treating ocular neovascularization, characterized in that said compound is at a substantially neutral pH in a pharmaceutically acceptable form suitable for delivery to the eye.

As used herein the phrase “substantially neutral pH” refers to a pH that is between about 5 and about 9 and would include pH's such as 5.5, 6, 6.5, 7, 7.5, 8, and 8.5 and variations in such pH's. Where the phrase is used in conjunction with a formulation that is to be injected into the ocular environment the phrase will have the additional limitation that the final formulation for administration will be at or about a level that is substantially compatible with the ocular environment.

The concentration of heparin employed in the formulation ranges from about 0.01 pg/ml to about 100 mg/ml. Preferably heparin is administered in a substantially non-toxic amount or concentration, which may depend on the route of administration, the compound employed and a host of patient related factors.

Heparin is a heterogeneous group of straight-chain anionic mucopolysaccharides, termed glycosaminoglycans, having anticoagulant activity. The primary sugars are a-L-iduronic acid 2-sulfate, 2-deoxy-2-sulfamino-α-Dglucose 6-sulfate, (3-D-glucuronic acid, 2-acetamido-2-deoxy-a-D-glucose, and α-L-iduronic acid. These sugars are present in different amounts and are joined by glycosidic linkages, forming polymers of varying sizes. Heparin is strongly acidic because of its content of covalently linked sulfate and carboxylic acid groups. In heparin sodium, the acidic protons of the sulfates are partially replaced by sodium ions. In one embodiment of the invention, low molecular weight heparin is used. Low molecular weight heparin is derived from standard heparin through either chemical or enzymatic depolymerization, and is commercially available. Standard heparin has a molecular weight of about 5,000 daltons to about 30,000 daltons, while low molecular weight heparin has a molecular weight of about 1,000 daltons to about 10,000 daltons. Compared to standard heparin, low molecular weight heparin binds less strongly to protein, has enhanced bioavailability, interacts less with platelets and yields a predictable dose response and dose-dependent plasma levels, and produces less bleeding for a given antithrombotic effect. Low molecular weight heparin may be heparin sulfate, a lower-sulfated, higher-acetylated form of heparin. All of these are commercially available (e.g., Sigma Aldrich, St. Louis Mo.).

A possible mechanism for the beneficial effect of heparin or low molecular weight heparin in reducing vessel growth and proliferation is its polyanionic structure, which readily binds to polycationic angiogenic factors. Angiogenic factors with heparin bound to them have reduced biological activity, and therefore do not promote new vessel growth. In vivo, heparin sulfates are bound to the extracellular matrix (ECM) and endothelial cell surfaces. Heparin sulfate in the ECM may have a role in storing active growth factors that can be released when needed to exert immediate effects. Soluble heparins compete with heparin sulfates on the ECM for growth factors and proteins, and may consequently cause their release. Unfractionated heparin (UFH) may cause an increase in the plasma level of growth factors. Unlike UFH, which may promote angiogenesis, low molecular weight heparin may hinder the binding of growth factors to their high affinity receptors as a result of its smaller size. Low molecular weight heparin may affect the injured neovascular cornea by binding angiogenic factors that have been released from the ECM, as well as competitively (antagonistically) binding to angiogenic receptors.

In one embodiment, the concentration of heparin or low molecular weight heparin ranges from about 0.01 pg/ml to about 30 mg/ml. Alternatively, heparin or low molecular weight heparin may be administered in a concentration ranging from about 1 mg/ml to about 10 mg/ml. In a more preferred form of the invention, the concentration of heparin or low molecular weight heparin ranges from about 0.5 mg/ml to about 15 mg/ml to 20 mg/ml (for example, administration of 0.1 ml of a 100 mg/ml formulation of low molecular weight heparin). In various embodiments, the concentration may be about 0.5 mg/ml to about 2.5 mg/ml, about 1 mg/ml to about 5 mg/ml, about 5 mg/ml to about 10 mg/ml, or about 5 mg/ml to about 30 mg/ml. Any concentration within these ranges may be used.

In another embodiment the invention resides in an ocular pharmaceutically acceptable formulation (that is, containing buffers and excipients as known to one skilled in the art) which comprises: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization; and (b) at least a compound in a concentration and dose sufficient to reduce ocular neovascularization wherein the compounds are selected from the group consisting of: a tetracycline or a derivative thereof (including CMTs which inhibit MMP activity), a steroid, an antimicrobial, an anti-prostaglandin, and/or a metalloproteinase inhibitor.

In one form, formulations of the invention comprise a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) in a concentration from about 0.01 pg/ml to about 30 mg/ml.

As used herein a tetracycline or a derivative thereof including CMTs which inhibit matrix metalloproteinase (MMP) activity will include, but not limited to, inter alia: doxycycline, demeclocycline, minocycline, oxytetracycline, lymecycline, or a chemically modified tetracycline. Chemically modified tetracyclines (CMT) include demeclocycline, minocyciine, oxytetracycline and like compounds that inhibit the synthesis of MMP-8 and MMP-9. These include CMT such as CMT-315, CMT-3, CMT-8, and CMT-308; 6-demethyl-6-deoxy-4-dedimethylaminotetracylcine (COL-3), and others, for example, as described by Liu et al., A Chemically Modified Tetracycline (CMT-3) Is a New Antifungal Agent in Antimicrobial Agents Chemother. 2002 May; 46:1447; Seftor et al., Targeting the Tumor Microenvironment with Chemically Modified Tetracyclines: Inhibition of Laminin 5γ2 Chain Promigratory Fragments and Vasculogenic Mimicry 2002 November; 1: 1173, which are expressly incorporated by reference herein.

Tetracyclines exert their biological effects independent of their antimicrobial activity. That is, they inhibit MMPs and can prevent pathogenic tissue destruction. Furthermore, recent studies have suggested that tetracyclines and inhibitors of metalloproteinases suppress tumor progression, bone resorption, and angiogenesis and may have anti-inflammatory properties. Thus, a possible mechanism for the beneficial effect of tetracyclines and like compounds in reducing vessel growth and proliferation in the ocular region is via inhibition of metalloproteinases, which are zinc-dependent proteinase enzymes associated with the tumorigenic process. Selective inhibition of such metalloproteinase by the inventive formulations and methods described herein is believed to inhibit reactions leading to ocular neovascularization. Such metalloproteinase inhibitors are also included in the invention.

In a highly preferred form of the invention, doxycycline is the tetracycline derivative employed in the formulation. Doxycycline (4-(dimethylamino)-1,4,4a,5,5a,6,11,12a-octahydro3,5,10,12,12a-pentahydroxy-6-methyl-1,11-dioxo-2-naphthacenecarboxamide monohydrate, C₂₂H₂₄N₂O₈.H₂O) is a broad spectrum antibiotic in the class of tetracycline antibiotics. It is commercially available.

According to this form of the invention the formulation comprises doxycycline in an amount sufficient to reduce ocular neovascularization together with excipients for topical, subconjunctival, or intraocular administration. For example the formulation might contain 2% doxycycline at a substantially neutral pH.

The concentration of doxycycline employed in this form of the invention will range from 0.01 μg/ml to about 30 mg/ml. More specifically, doxycycline concentrations will range from about 0.05 mg/ml to about 1 mg/ml. Alternatively doxycycline concentrations will range from about 0.05 mg/ml to about 10 mg/ml. Yet again doxycycline concentrations can range from about 1 mg/ml to about 20 mg/ml. These doses are substantially non-toxic to the patient. Besides its anti-angiogenic effect, doxycycline could reduce the incidence of endophthalmitis, which occurs in about 0.5% of eyes in which a steroid is administered.

In a highly preferred form the following formulations may be used: a 1:1 combination of about 20 mg/ml doxycycline and about 10 mg/ml heparin or low molecular weight heparin (actual concentration 10 mg/ml doxycycline with 5 mg/ml heparin or low molecular weight heparin).

In second form formulations of the invention comprise a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid at a concentration from about 0.1 mg/ml to about 40 mg/ml.

Steroids are usually administered for ocular pathologies such as uveitis, diabetic retinopathy, idiopathic juxtafoveal telangiectasias, macular edema secondary to diabetes mellitus, central retinal vein occlusion, pseudophakia, during photodynamic therapy for age related macular degeneration, etc., and for intraoperative visualization of the posterior hyaloid, which also desirably inhibit ocular neovascularization. An undesirable and serious side effect of ocular steroid therapy is increased intraocular pressure, termed glaucoma or ocular hypertension. For patients with glaucoma or predisposed to glaucoma, steroid therapy presents a risk for unacceptably high intraocular pressure, such that surgery may be required to lower the intraocular pressure. Such risks and benefits must be balanced in determining whether to treat the patient with triamcinolone or other steroids. The formulations and methods to predict patients at risk for glaucoma from steroid therapy, disclosed in co-pending U.S. patent application Ser. No. 10/787,580, which is expressly incorporated by reference herein in its entirety.

Steroids for ocular administration include, but are not limited to, triamcinolone (Aristocort®; Kenalog®), betamethasone (Celestone®), budesonide, cortisone, dexamethasone (Decadron-LA®; Decadron® phosphate; Maxidex® and Tobradex® (Alcon)), hydrocortisone, methylprednisolone (DepoMedrol®, Solu-Medrol®), prednisolone (prednisolone acetate, e.g., Pred Forte® (Allergan); Econopred and Econopred Plus® (Alcon); AK-Tate® (Akorn); Pred Mild® (Allergan); prednisone sodium phosphate (Inflamase Mild and Inflamase Forte® (Ciba); Metreton® (Schering); AK-Pred® (Akorn)), fluorometholone (fluorometholone acetate (Flarex® (Alcon); Eflone®), fluorometholone alcohol (FML® and FML-Mild®, (Allergan); Fluor OP®)), rimexolone (Vexol® (Alcon)), medrysone alcohol (HMSO (Allergan)); lotoprednol etabonate (Lotemax® and Alre)®(Bausch & Lomb), 11-desoxcortisol, and anecortave acetate (Alcon)). It will be appreciated that the above lists are representative only and are not exclusive.

In a highly preferred form of the invention the steroid used in the formulation is a 11-substituted-16α,17α-substituted methylenedioxy steroid selected from the compounds disclosed in U.S. Pat. No. 5,770,589 to Billson and Penfold (“US '589”), which was filed as U.S. application Ser. No. 08/586,750, and is incorporated herein in its entirety by reference. Alternatively the compound is a steroid disclosed in Fried et al. (1958) J. Am. Chem. Soc. 80, 2338 (1958); U.S. Pat. No. 2,990,401; U.S. Pat. No. 3,048,581 or U.S. Pat. No. 3,035,050 each of which is expressly herein incorporated by reference. Collectively these publications also provide methods for the manufacture of such compounds and are also incorporated for the purposes of disclosing such methods. Desirably the steroid used in the method is triamcinolone acetonide.

The steroid concentration in the inventive formulation ranges from about 0.1 mg/ml to about 40 mg/ml. More preferably the steroid concentration ranges from about 1 mg/ml to about 20 mg/ml. Alternatively, steroid concentrations range from about 20 mg/ml to about 30 mg/ml or they can range from about 20 mg/ml to about 40 mg/ml.

The steroid concentration used with a particular formulation will depend upon the particular steroid that is used. For example, triamcinolone acetonide (9α-fluoro-11 13,16a,17,21 tetra hydroxy-pregna-1,4diene-3,20-dione cyclic 16,17-acetal with acetone (C₂₄H₃₁FO₆)) Kenacort®), Kenalog® (Bristol-Myers Squibb, Princeton N.J.) may be administered at a therapeutic dose in the range of about 4 mg to about 8 mg, for example, by intravitreous injection. In comparison, anecortave acetate, a steroid with less potential to cause an increase in intraocular pressure than triamcinolone but not used inside the eye, may be administered at dose of about 0.5 mg/ml to about 30 mg/ml.

In a third form, formulations of the invention comprise a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an anti-prostaglandin in a concentration from about 1 μg/ml to about 10 mg/ml (such as a 1 μg/ml to about 10 mg/ml dose of flurbiprofen).

Anti-prostaglandins, also termed prostaglandin antagonists, may be administered in a concentration sufficient to result in a prostaglandin-inhibitory effect. As one example, antiprostaglandins such as flurbiprofen may be administered at a concentration in the range of about 0.001%^{w/v} to about 0.5%^{w/v}. As an example, OCUFEN® (flurbiprofen sodium 0.03% (Allergan), sodium (±)-2-(2-fluoro-4-biphenylyl)-propionate dihydrate) 0.03% may be administered at a concentration ranging from about 0.003%^{w/w} to about 0.3%^{w/w}. Anti-prostaglandins other than flurbiprofen may be included. The anti-prostaglandins maybe administered at the doses and by the methods previously described, and include indomethacin, ketorolac, tromethamine 0.5% ((±)-5-benzoyl-2,3-dihydro-1H-pyrrolizine-1-carboxylic acid, compound with 2-amino-2-(hydroxymethyl)-1,3-propanediol (1:1) (ACULAR® Allegan, Irvine Calif.), meclofenamate, fluorbiprofen, and compounds in the pyrrolo-pyrrole group of non-steroidal anti-inflammatory drugs (NSAIDs). For example, ACUALR® may be administered at a concentration ranging from about 0.003%^{w/w} to about 0.3%^{w/w}. In one embodiment, the concentration of ACULAR® is about 0.03%^{w/w}.

In a fourth form, formulations of the invention comprise a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a antimicrobial, like for example a macrolide antibiotic, in a concentration from about 20 μg/ml to about 200 μg/ml (about 0.002%^{w/v} to about 0.02%^{w/v}).

A possible mechanism for the beneficial effect of macrolide antibiotics are their anti-inflammatory effect.

Macrolide antibiotic that can be added to the inventive formulation include, but are not limited to, inter alia: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide. Other antibiotics include, but are not limited to, aminoglycosides (e.g., streptomycin, amikacin, gentamicin, tobramycin), cephalosporins (e.g., beta lactams including penicillin), tetracyclines, acyclorvir, amantadine, polymyxin B, amphtotericin B, amoxicillin, ampicillin, atovaquone, azithromycin, azithromycin, bacitracin, cefazolin, cefepime, cefotaxime, cefotetan, cefpodoxime, ceftazidime, ceftizoxime, ceftriaxone, cefuroxime, cephalexin, chloramphenicol, clotimazole, ciprofloxacin, clarithromycin, clindamycin, dapsone, dicloxacillin, fluconazole, foscarnet, ganciclovir, gatifloxacin, griseofulvin, isoniazid, itraconazole, ketoconazole, metronidazole, nafcillin, neomycin, nitrofurantoin, nystatin, pentamidine, rifampin, rifamycin, valacyclovir, vancomycin, etc. The indications, effective doses, formulations, contraindications, vendors, etc. of these antibiotics are known to one skilled in the art.

Macrolide antibiotics can be administered in a concentration ranging from about 20 μg/ml to about 200 μg/ml (about 0.002%^{w/v} to about 0.02%^{w/v}). Formulations and doses of macrolide antibiotics are described in co-pending U.S. patent application Ser. Nos. 10/667,161 and 10/752,124, each of which is expressly incorporated by reference herein in its entirety.

In addition to a macrolide antibiotic the formulation can also include mycophenolic acid. Such a formulation when prepared as a pharmaceutically acceptable topically administered solution may include about 0.5%^{w/v} to about 10%^{w/v} mycophenolic acid. Preferably, a concentration of macrolide antibiotic and/or mycophenolic acid in a pharmaceutically acceptable topically administered solution may range from about 3%^{w/v} to about 5%^{w/v}. In another embodiment, a concentration of macrolide antibiotic and/or mycophenolic acid in a pharmaceutically acceptable topically administered solution may range from about 1%^{w/v} to about 3%^{w/v}. In another embodiment, a concentration of macrolide antibiotic and/or mycophenolic acid in a pharmaceutically acceptable topically administered solution may range from about 3%^{w/v} to about 10%^{w/v}. In another embodiment, a concentration of macrolide antibiotic and/or mycophenolic acid may range from about 0.1% to about 10% in a topical ocular formulation for treating diabetic retinopathy, age related macular degeneration, or retinitis pigmentosa. In another embodiment, concentrations of macrolide antibiotics and/or mycophenolic acid up to about 2%, up to about 5%, up to about 10%, or exceeding 10% are formulated for topical administration when the compound(s) is bound to a matrix or polymer which slowly releases the compound(s) over time while not exceeding an intraocular concentration of 40 μg/ml.

In a fifth form, the formulation comprises a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an inhibitor of a metalloproteinase in a concentration and dose to reduce ocular neovascularization.

Inhibitors of metalloproteinases include naturally occurring proteins such as TIMP-1 that specifically inhibit matrix metalloproteinases (MMPs), and synthetic metalloproteinase inhibitors such as Batimastat (BB-94) and marimastat (BB-2516) which potently and specifically inhibit metalloproteinase production. These inhibitors degrade the extracellular matrix, promoting tumor invasion and metastasis, but also regulate host defense mechanisms and normal cell function. Selective inhibition is expected to inhibit reactions leading to neovascularization in the inventive formulations and methods. Such metalloproteinase inhibitors are also included in the invention. Among the twenty-four MMPs described, eight have been identified in the cornea, i.e., collagenase I and III (MMP-1 and MMP-13), gelatinase A and B (MMP-2 and -9), stromelysin (MMP-3), matrilysin (MMP-7) and membrane type MMP (MMP-14).

In an alternate embodiment of the invention the formulation comprises: (a) a heparin or low molecular weight heparin in a concentration from about 0.1 mg/ml to about 100 mg/ml, characterized in that said compound is at a substantially neutral pH in a pharmaceutically acceptable form suitable for delivery to the eye to reduce ocular neovascularization; and (b) a plurality of compounds in a concentration and dose to reduce ocular neovascularization, wherein the compounds are selected from the group consisting of: a tetracycline or a derivative thereof (including CMTs which inhibit MMP activity), a steroid, an antimicrobial, an anti-prostaglandin, and/or a metalloproteinase inhibitor.

Where there are a plurality of tetracycline compounds or a derivative thereof (including CMTs which inhibit MMP activity), steroids, antimicrobials, anti-prostaglandins, and/or a metalloproteinase inhibitor compounds employed in the formulation, the preferred compounds and their doses will be those which are described above. In an illustrative form of the invention such a formulation can comprise:
(1) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml;
(2) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an anti-prostaglandin such as flurbiprofen at a concentration from about 1 μg/ml to about 10 mg/ml;
(3) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a macrolide antibiotic such as ascomycin at a concentration from about 20 μg/ml to about 200 μg/ml;
(4) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an anti-prostaglandin such as flurbiprofen at a concentration from about 1 μg/ml to about 10 mg/ml and an macrolide antibiotic such as ascomycin at a concentration from about 20 μg/ml to about 200 μg/ml;
(5) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an anti-prostaglandin such as flurbiprofen at a concentration from about 1 μg/ml to about 10 mg/ml and an inhibitor of a metalloproteinase in a concentration and dose to reduce ocular neovascularization;
(6) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an inhibitor of a metalloproteinase in a concentration and dose to reduce ocular neovascularization;
(7) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml; or
(8) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and a macrolide antibiotic such as ascomycin at a concentration from about 20 μg/ml to about 200 μg/ml; or
(9) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml and a macrolide antibiotic such as ascomycin at a concentration from about 20 μg/ml to about 200 μg/ml.

The skilled reader will appreciate that the duration over which any of the formulations of the invention will dwell in the ocular environment will depend, inter alia, on such factors as the pharmacological properties of the compounds employed in the formulation, the concentration of the compound employed, the bioavailability of the compound, the disease to be treated the mode of administration and the preferred longevity of the treatment. Where that balance is struck will often depend on the longevity of the effect required in the eye and the ailment being treated. Formulations prepared according to the invention will preferably have dwell times from hours to many months and possibly years, although the latter time period requires special delivery systems to attain such a duration. Some illustrative forms of such delivery systems are described below. Most preferably the formulations described herein will have a dwell time (ie duration in the eye) of hours (i.e. 1 to 24 hours), days (i.e. 1, 2, 3, 4, 5, 6 or 7 days) or weeks (i.e. 1, 2, 3, 4 weeks). Alternatively, the formulation will have a dwell time of at least a few months such as, 1 month, 2 months, 3 months, with dwell times of greater than 4, 5, 6, 7 to 12 months being achievable.

The precise formulation used in the pharmaceutical formulation of the present invention will vary according to a wide range of commercial and scientific criteria. That is the skilled reader will appreciate that the above formulation of the invention described above may contain other agents. For example the formulations of the invention are preferably prepared using a physiological saline solution as a vehicle. The pH of the formulation may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulation may additionally include at least a pharmaceutically acceptable additive (such as a diluent, carrier, adjunct, excipient or non-toxic, non-therapeutic, non-immunogenic stabilizers and the like). Preferably, the pharmaceutically acceptable additive should be ophthalmologically acceptable, preferably being compatible with the vitreous, and should not leave any vision impairing residue in the eye. Desirably, any pharmaceutically acceptable additive used in the formulation may preferably be suited to the delivery of said pharmaceutical formulation as an intravitreal depot injection. Any diluent used in the preparation of the pharmaceutically acceptable formulation may preferably be selected so as not to unduly affect the biological activity of the formulation. Examples of such diluents which are especially useful for injectable formulations are water, the various saline, organic or inorganic salt solutions, Ringer's solution, dextrose solution, and Hank's solution.

In addition, the pharmaceutical formulation may include additives such as other buffers, diluents, carriers, adjuvants or excipients. Any pharmacologically acceptable buffer suitable for application to the eye may be used, e.g., tris or phosphate buffers. Other agents may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. The indications, effective doses, formulations, contraindicatons, vendors etc, of the compounds in the formulations are available or are known to one skilled in the art.

These agents may be present in individual amounts of from about 0.001 to about 5% by weight and preferably about 0.01% to about 2%. Suitable water soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 9 and preferably about 4 to about 8. As such the buffering agent may be as much as about 5% on a weight to weight basis of the total formulation. Electrolytes such as, but not limited to, sodium chloride and potassium chloride may also be included in the formulation.

Any of the formulations may be administered by an ocular route, such as topical, subconjunctival, sub-Tenon, intraocular, etc. Moreover the formulation may be administered as a slow release formulation, with a carrier formulation such as nanospheres, nanocapsules, microspheres, microcapsules, liposomes, etc., as an intravenous solution or suspension, or in an intraocular injection, as known to one skilled in the art. A time-release drug delivery system may be administered intraocularly to result in sustained release of the agent over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered through a needle, or may be implanted by suturing within the eye, for example, within the lens capsule. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, New York 2000), the relevant sections of which are incorporated by reference herein in their entirety. For example, a sustained release intraocular implant may be inserted through the pars plana for implantation in the vitreous cavity. An intraocular injection may be into the vitreous (intravitreal), or under the conjunctiva (subconjunctival), or behind the eye (retrobulbar), or under the Capsule of Tenon (sub-Tenon), and may be in a depot form. Other intraocular routes of administration and injection sites and forms are also contemplated and are within the scope of the invention.

Administration of the inventive formulation should at least reduce ocular neovascularization. Vessel regression may occur in addition to, or in place of, prevention of further vessel growth or proliferation. As will be appreciated, the cumulative effects may be important in managing diseases such as diabetes, where control of the complicating factors of the disease is as important as control of the underlying pathology to maintain a patient's quality of life.

Accordingly in another embodiment, the invention resides in a method for reducing ocular neovascularization comprising the step of: administering to a patient a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization. In a more preferred form of the invention the formulation used in the above method is a formulation described above to reduce neovascularization in the anterior and/or posterior portions of the eye, or in the cornea, retina, choroid, etc. The route and form of administration of the heparin formulation may be any method known to one skilled in the art, and as previously described. Administration may be by topical, subconjunctival, and intraocular routes or ocular implants.

In one embodiment, the formulation is intraocularly injected, for example, into the vitreous. When administering the formulation by intravitreal injection, the active agents should be concentrated to minimise the volume for injection. For injection, a concentration less than about 20 mg/ml may be injected, and any amount may be effective depending upon the factors previously described. Preferably a dose of less than 7 mg/ml is administered, with doses of less than 6 mg/ml, 5 mg/ml, 4 mg/ml 3 mg/ml, 2 mg/ml and 1 mg/ml being more preferred. Sample concentrations include, but are not limited to, about 5 μg/ml to about 50 μg/ml; about 25 μg/ml to about 100 μg/ml; about 100 μg/ml to about 200 μg/ml; about 200 μg/ml to about 500 μg/ml; about 500 μg/ml to about 750 μg/ml; about 500 μg/ml up to 1 mg/ml; etc.

For example, in preparation for injection, topical alcaine was applied to the ocular surface, followed by 5% povidone iodine. A cotton-tipped applicator soaked in 4% lidocaine was then applied to the injection site, which is 4.0 mm posterior to the limbus in phakic eyes and 3.5 mm posterior to the limbus in pseudophakic eyes. A 27-gauge needle was used for injection at the superior pars plana. Indirect ophthalmoscopy can be used to confirm proper intravitreal placement of the suspension.

The syringe used in practicing this invention is suitably one which can accommodate a 21 to 30 gauge needle (eg a 23, 24, 25, 26 or 27 gauge needle) and is preferably of a small volume, for example 1.5 mL, or more preferably 0.5 mL. Although it is possible that the needle and syringe may be of the type where the needle is removable from the syringe, it is preferred that the arrangement is of a unitary syringe/needle construction. This would clearly limit the possibility of disengagement of the needle from the syringe. It is also preferred that the arrangement be tamper evident. The formulations of the present invention may therefore be provided in the form of a single unit dose in a pre-prepared syringe, ready for administration.

A suitable style of syringe is, for example, sold under the name of Uniject™ manufactured by Becton Dickinson and Company. In this style of syringe, the material is expelled through the needle into the eye by pressure applied to the sides of a pliable reservoir supplying the needle, rather than by a plunger. As the name implies, the construction of the reservoir and needle forms a single unit.

Topical application of formulations of the invention may be as an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye. Indeed, it can be advantageous to formulate a formulation of the invention as a gel, to minimize loss of the formulation immediately upon administration, as a result, for example, of lacrimation caused by reflex blinking. Although it is preferred that such a formulation exhibit further increase in viscosity or gel stiffness upon administration, this is not absolutely required if the initial gel is sufficiently resistant to dissipation by lacrimal drainage to provide the effective residence time specified herein.

To prepare a topical formulation for the treatment of ophthalmological disorders, a therapeutically effective amount of the formulation of the invention is placed in an ophthalmological vehicle as is known in the art. The amount of the therapeutic compound to be administered and the concentration of the compound in the topical formulations depend upon the diluent, delivery system or device selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

For topical administration, the concentration of heparin administered may depend upon the particular patient, the underlying disease and its severity, the dosing frequency, etc., as known to one skilled in the art. Sample concentrations include, but are not limited to, about 0.5 mg/ml to about 2.5 mg/ml, about 1 mg/ml to about 5 mg/ml, about 5 mg/ml to about 10 mg/ml, about 10 mg/ml to about 15 mg/ml, about 15 mg/ml up to 30 mg/ml, etc.

Where the formulation contains two or more active agents, the active agents may be administered as a mixture, as an admixture, in the same formulation, in separate formulations, in extended release formulations, liposomes, microcapsules, or any of the previously described embodiments.

The formulation may be administered topically, or may be injected into the eye, or one active agent may be administered topically and the other agent(s) may be injected.

The method of the present invention may be performed alone, or in combination with one or more other therapies such as photodynamic therapy, laser treatment, or one or more biological or pharmaceutical treatments.

In another embodiment the invention resides in a method for reducing ocular irritation comprising the step of: administering to a patient a formulation as described above to a patient following corneal surgery (e.g., LASIK® surgery, photorefractive keratectomy (PRK), or other corneal procedures). Preferably the formulation administered to the patient is a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof including CMTs which inhibit MMP activity or a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an antiprostaglandin such as flurbiprofen. Alternatively an anti-prostaglandin agent may be administered with a tetracycline or a derivative thereof including CMTs which inhibit MMP activity and a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization.

In a yet another embodiment of the method of the invention, one or more of the formulations described above is administered to a patient in a cyclic tumor treatment regimen to reduce blood vessel growth and proliferation at a tumor site. In this form of the invention, the agents are systemically administered along with standard tumor therapies, so that the agents are rotated, thereby inhibiting blood vessel proliferation throughout the treatment cycle.

In this embodiment, the initial therapy (stage 1) is selected among those presently available: either chemotherapy (e.g., gene therapy, antineoplastic drugs, etc.) or one or more of the following non-chemotherapeutic treatments: radiation therapy (e.g, x-rays, gamma rays, (3 rays, etc.); phototherapy (e.g., photodynamic therapy, photosensitizers); or thermal therapy (e.g., thermal coagulation, hyperthermia, cryotherapy).

Immediately following this initial treatment event, therapy using the inventive formulations is initiated in a rotational cycle. That is, one or more of the formulations described above is administered over the course of one cycle, but the active agents are administered at different stages in the cycle. Each of the agents is administered systemically (e.g., intravenously, orally, etc.) at their highest nontoxic concentration, as known to one skilled in the art. For example, steroids are administered at doses ranging from about 100 mg/ml to about 200 mg/ml. The use of a cyclic rotational administration of each of these vessel-inhibiting agents causes vessel damage at different times and through different processes, thereby maximizing the overall damage to the vessels and inhibiting blood supply to the tumor while conventional tumor therapy occurs (e.g., chemotherapy, radiation therapy, etc.).

The inventive cyclic therapy is initiated by systemic administration of a steroid, followed by systemic administration of a formulation containing the same or another steroid and a heparin or low molecular weight heparin in a concentration from about 0.01 pg/ml to about 100 mg/ml (stage 2). For example intravenous administration of methylprednisolone (Solu-Medrol®) can be followed by oral administration of prednisone and doxycycline. Stage 2 lasts from about one to about two weeks. Stage 3 follows stage 2, during which a formulation containing a heparin or low molecular weight heparin in a concentration from about 0.01 pg/ml to about 100 mg/ml and heparin is administered. Chemotherapeutic drugs may also be administered in stage 3. Stage 3 lasts from about one to about two weeks. Stage 4 follows stage 3, during which a formulation containing a heparin or low molecular weight heparin in a concentration from about 0.01 pg/ml to about 100 mg/ml, anti-prostaglandins, and macrolide antibiotics are administered. Stage 4 lasts from about one to about two weeks and completes the first treatment cycle, which lasts from about one to about two months.

If additional therapy is required (determined by tumor size, the presence of absence of tumor markers, etc.), further cycle(s) of treatment are initiated. These further cycles may start either with stage 1 and proceed through stages 2, 3, and 4, or may start with stage 2 directly from stage 4 and bypass stage 1. It will be appreciated that any of the agents described herein may be used in any of stages 2, 3, or 4.

In addition to the above other substances, formulations of the invention may be injected with anti-angiogenic agents designed to block the actions of VEGF on endothelial cells that can be employed in the method of the invention are: (a) Lucentis® made by Genentech; and (b) Macugen® made by Eyetech Pharmaceuticals. Lucentis® and Macugen® are compounds that are injected into the vitreous and are potent anti-angiogenic compounds. In a highly preferred form, the pharmaceutical formulation of the invention will comprise a formulation of the invention as described and an anti-angiogenic agent such as Lucentis® or Macugen®.

Lucentis® (ranibizumab), formerly known as rhuFab V2 or AMD-Fab is a humanized, therapeutic anti-VEGF (vascular endothelial growth factor) antibody fragment developed at Genentech to bind and inhibit VEGF, a protein that plays a critical role in angiogenesis (the formation of new blood vessels). Lucentis is designed to block new blood vessel growth and reduce leakage, which are thought to lead to wet AMD disease progression. When administered in conjunction with pharmaceutical formulations prepared according to the present invention Lucentis should be administered in either about 300 or about 500 microgram doses for four doses.

Macugen® (pegaptanib sodium, anti-VEGF aptamer or EYE001) made by Eyetech Pharmaceuticals, consists of a synthetic fragment of genetic material that specifically binds to the VEGF molecule and blocks it from stimulating the receptor on the surface of endothelial cells. When administered in conjunction with pharmaceutical formulations prepared according to the present invention Macugen® should be administered in a dose ranging from either about 0.3 mg to about 3.0 mg every four or six weeks.

In another aspect of the invention pharmaceutical formulations prepared according to the present invention may be prepared in combination with a glucocorticoid (e.g. prednisolone, prednisone), an oestrogen (e.g. oestrodiol), an androgen (e.g. testosterone) retinoic acid derivatives (e.g. 9-cis-retinoic acid, 13-trans-retinoic acid, all-trans retinoic acid), a vitamin D derivative (e.g. calcipotriol, calcipotriene), a non-steroidal anti-inflammatory agent, a vitamin D derivative, an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein. In still other embodiments of the invention, a mixture of these agents may be used.

Ocular anti-infective agents as described herein include, but are not limited to, penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (amikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscarnet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan), anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), immunosuppressant agents such as cyclosporin A and SK506, and anti-inflammatory or suppressive factors (inhibitors), and inhibitors of proteolytic enzymes such as plasminogen activator inhibitors. Doses for topical and sub-conjunctival administration of the above agents, as well as intravitreal dose and vitreous half-life may be found in Intravitreal Surgery Principles and Practice, Peyman G A and Shulman, J Eds., 2nd edition, 1994, Appleton-Longe, the relevant sections of which are expressly incorporated by reference herein.

Use a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization in the manufacture of a medicament for the treatment of ocular neovascularization.

Use of a formulation as herein described in the preparation of a medicament for the treatment of ocular neovascularization.

Use of a formulation as herein described as well as anti-angiogenic agents designed to block the actions of VEGF on endothelial cells in the preparation of a medicament for the treatment of ocular neovascularization.

### EXAMPLES

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

### Example 1

Artificial corneal burns were induced in rat eyes to determine the effects of doxycycline, steroids, and low molecular weight heparin, alone and in combinations, on corneal neovascularization. More specifically, topical administration of doxycycline, low molecular weight heparin, and triamcinolone were administered twice a day to rats in which corneal burns had been artificially induced by application of silver nitrate (70%) and potassium nitrate (30%).

The presence of new vessels (neovascularization) and the extent of new vessel formation was assessed by split lamp photography and histology. Inhibition of vessel proliferation was evaluated by measuring vessel progression from the outer cornea (corneal limbus) into the cornea. A numerical rating system was used to quantitate the degree of inhibition (+, ++, and +++ inhibition), with “+ inhibition” indicating inhibition one-third of the distance from the limbus of the cornea to the center; “++ inhibition” indicating inhibition two-thirds of the distance from the limbus to the center; “+++ inhibition” indicating complete inhibition of vessels between the limbus and the center; and the designation “± inhibition” indicating an intermediate degree of inhibition (e.g, less than +, ++, or +++). As previously described, it will be appreciated that any reduction of new vessel proliferation and/or regression of existing vessels is therapeutic, and that complete inhibition and/or regression is not required, and also that reduction includes regression of existing vessels.

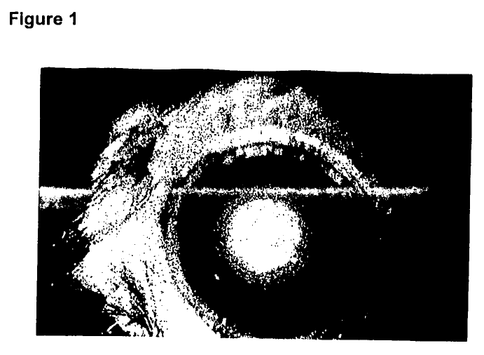

Full vascularization was seen after one week of saline administration (control), as seen in FIG. 1. Any of the above agents alone, when topically applied to affected corneas, did not completely inhibit neovascularization. For example, corneas treated with topically applied doxycycline at a concentration of about 1 mg/ml to about 20 mg/ml showed + inhibition of neovascularization compared to controls. Corneas treated with topically applied low molecular weight heparin at a concentration of about 10 mg/ml showed + inhibition of neovascularization compared to controls. Corneas treated with topically applied triamcinolone at a concentration of about 4 mg/ml showed ++ inhibition of neovascularization.

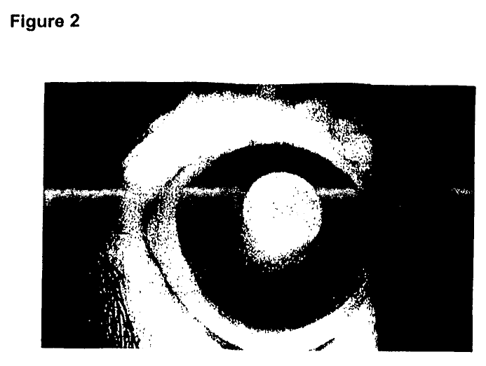

In contrast, when a formulation of doxycycline (about 20 mg/ml) and triamcinolone (4 mg/ml) was topically applied to the affected cornea twice a day, there was +++ inhibition of neovascularization; that is, no neovascularization was evident. The +++ inhibition of new vessel growth was seen at one week after treatment, and the same +++ inhibition was maintained at three weeks, as shown in FIG. 2.

When a formulation of low molecular weight heparin (about 10 mg/ml) and triamcinolone was topically applied to the affected cornea twice a day, there was +++ inhibition of neovascularization after one week compared to the control eye. After three weeks, the inhibition of neovascularization was minimally diminished (++±) so that neovascularization inhibition was slightly less than the doxycycline and triamcinolone formulation applied, but there was still significant inhibition.

When a formulation of low molecular weight heparin (about 1 mg/ml) and doxycycline (about 20 mg/ml) was topically applied to the affected cornea twice a day, neovascularization was also inhibited after one week but to a lesser extent (++ to +++) compared to administration with either doxycycline and triamcinolone, or low molecular weight heparin and triamcinolone. After three weeks, there was still complete inhibition of neovascularization with doxycycline and low molecular weight heparin compared to controls. Neovascularization was not observed for the treatment duration.

### Example 2

The ability of the inventive formulation to cause regression of existing vessels was demonstrated. Neovascularization was induced over three days by topical application of a silver nitrate solution, as described in Example 1, to thirty-two rat eyes. Vascularization was allowed to proceed midway from the limbus to the cornea (days 1, 2, and 3).

On day 4, one dose of one of the following treatments was administered to the affected eyes (eight eyes per group): saline (control); a formulation of triamcinolone (40 mg/ml) and low molecular weight heparin (10 mg/ml); a formulation of doxycycline (20 mg/ml) and low molecular weight heparin (10 mg/ml); or a formulation of doxycycline (20 mg/ml) and triamcinolone (40 mg/ml). The same treatment regimen was repeated on each eye on both of days 5 and 6.

Eyes were examined on day 6. All of the control eyes showed vascular progression, in that the eyes were fully vascularized and no inhibition of vascularization occurred. That is, vascularization extended from the limbus to the cornea. In contrast, all the treated eyes, regardless of the treatment formulation, showed regression of Eyes treated with triamcinolone and low molecular weight heparin showed ++ to +++ reduced vascularization. Eyes treated with doxycycline and low molecular weight heparin showed + to ++ reduced vascularization. Eyes treated with doxycycline and triamcinolone showed ++ reduced.

### Example 3

Artificial corneal burns were induced in thirty-two eyes belonging to thirty-two Long Evans rats to determine the effects of doxycycline or another tetracycline derivative and low molecular weight heparin, doxycycline or another tetracycline derivative, and flurbiprofen, or flurbiprofen and low molecular weight heparin, on corneal neovascularization. All the eyes were examined to exclude any eyes with corneal scars and/or neovascularization prior to induction. More specifically, topical administration of the described two drug combination was administered twice a day to rats in which corneal burns had been artificially induced by application of silver nitrate (70%) and potassium nitrate (30%).

Neovascularization was induced in all eyes using silver nitrate cauterization. The animals were first anesthetized by intraperitoneal injection of a mixture of ketamine hydrochloride (25 mg/kg) with xylazine hydrochloride (5 mg/kg). The cornea was then anesthetized by a drop of 0.5% proparacaine and allowed to dry. One cornea of each animal was cauterized by pressing an applicator stick (diameter of 1.8 mm) coated with 75% silver nitrate/25% potassium nitrate (Arzol Chemical Co., Keen, N.H.) to the central cornea for ten seconds (using a stopwatch) under the operating microscope. Excess silver nitrate was removed by rinsing the eyes with balanced salt solution. To increase the reproducibility of the injuries, one investigator cauterized all animals.

Following cauterization, the animals were randomly divided into four groups to eliminate any potential bias in the degree of injury with the different groups. Group 1 (number of animals, n=8) received a 1:1 combination of 0.03% flurbiprofen sodium ophthalmic solution (Allergan, Irvine Calif.) and 10 mg/ml low molecular weight heparin (Enoxaparin, Aventis Pharmaceuticals Inc., Bridgewater N.J.); an actual concentration of 0.015% flurbiprofen with 5 mg/ml low molecular weight heparin. Group 2 (n=8) received a 1:1 combination of 0.03% flurbiprofen sodium ophthalmic solution and 20 mg/ml doxycycline (American Pharmaceutical Partners, Schaumburg Ill.); an actual concentration of 0.015% flurbiprofen with 10 mg/ml doxycycline. Group 3 (n=8) received a 1:1 combination of 20 mg/ml doxycycline and 10 mg/ml low molecular weight heparin; an actual concentration of 10 mg/ml doxycycline with 5 mg/ml low molecular weight heparin. Group 4 (n=8) received balanced salt solution (control). The drops were applied topically immediately after cauterization; treatments were administered two times per day for seven days.

The presence of new vessels (neovascularization) and the extent of new vessel formation was assessed by slit lamp photography and histology. Inhibition of vessel proliferation was evaluated by measuring vessel progression from the outer cornea (corneal limbus) into the cornea. As previously described, it will be appreciated that any reduction of new vessel proliferation and/or regression of existing vessels is therapeutic, and that complete inhibition and/or regression is not required, and also that reduction includes regression of existing vessels.

The extent of corneal neovascularization was determined by slit lamp microscopy with photography (SL-7E, Topcon, Tokyo Japan) on day seven after cauterization. The animals were euthanized in a carbon dioxide chamber under deep general anesthesia. The eyes were enucleated and fixed in 10% formaldehyde. After fixation for 24 hours, the eyes were removed from the fixative and corneas were dehydrated and sectioned. The corneas were then soaked in xylene and paraffin, later they were embedded in paraffin and cut at 1 pm for staining with hematoxylin-eosin (H&E) for light microscopy.

Corneal neovascularization was assessed by scanning (Cano scan 9900F, Canon, Tokyo Japan) the slit lamp photographs into high resolution digital images. The percentage area of corneal neovascularization was determined by outlining the areas with corneal vessels and comparing these to the total corneal surface using image j software (Wayne Rasband at the Research Services Branch, National Institute of Mental Health, Bethesda Md.). The percentage area of the cornea covered by the corneal scar in each eye was also determined. A drawing of corneal blood vessels was made to compare with digital photos and ensure that no vascular area was omitted during calculation of percent area.

Statistical analysis was performed using Statistical Analysis System (SPSS 11.5) software. The difference between the groups was determined using Mann-Whitney U Analysis test; a p value less than 0.05 was considered significant.

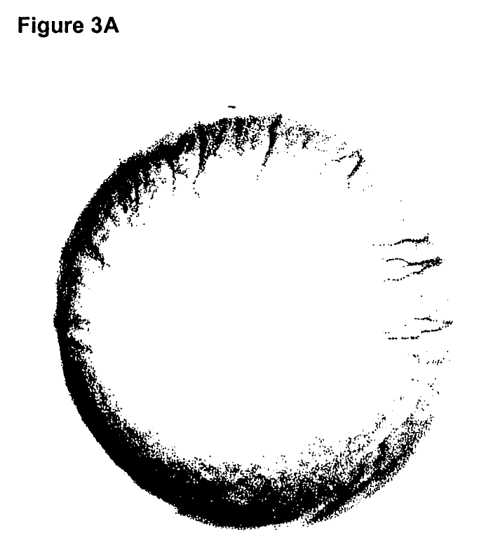

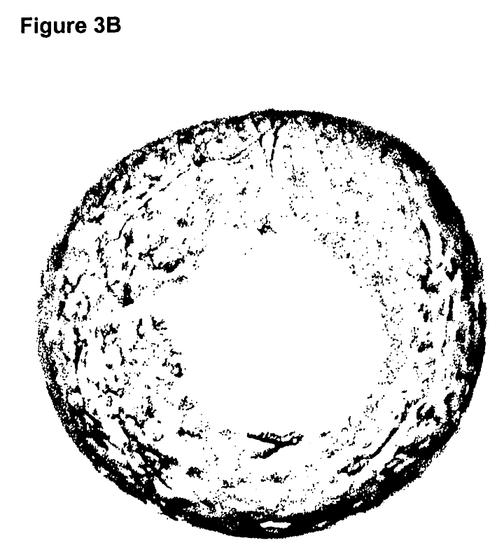

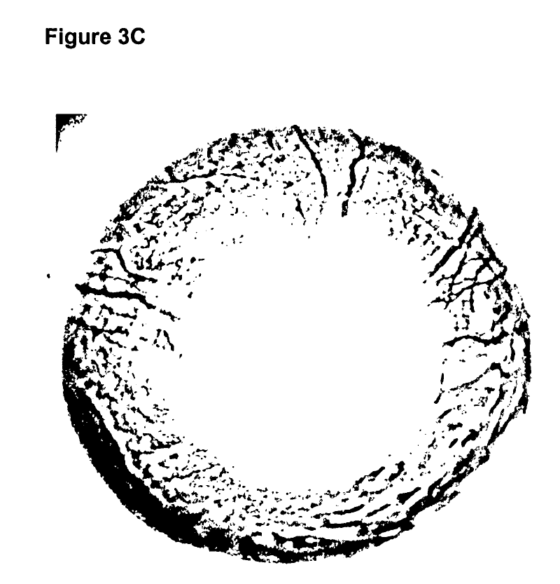

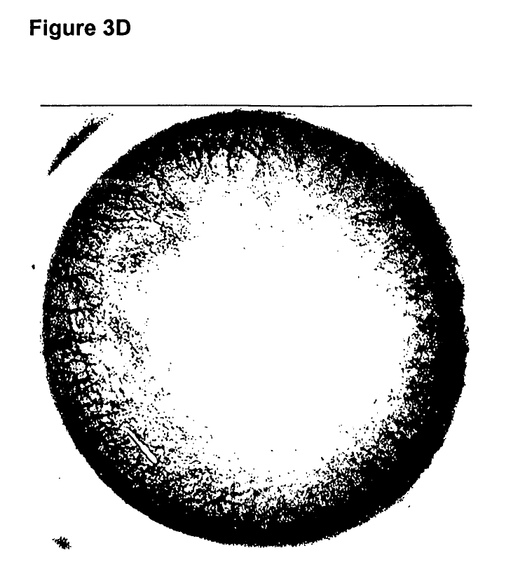

Representative digitally enhanced slit lamp photographs of the cornea seven days after induction of corneal burn in treated eyes are shown in FIGS. 3A-3D. After administration of flurbiprofen and low molecular weight heparin, neovascularization was prominent but was less than in the control group (FIG. 3A). After administration of flurbiprofen and doxycycline, there was minimal neovascularization (FIG. 3B). After administration of doxycycline and low molecular weight heparin there was moderate neovascularization (FIG. 3C). After administration of normal saline (control), there was extensive neovascularization (FIG. 3D).

The percentage of corneal neovascularization, corneal scar size and burn intensity was determined for all eyes using J image on the digitized slit lamp photographs.

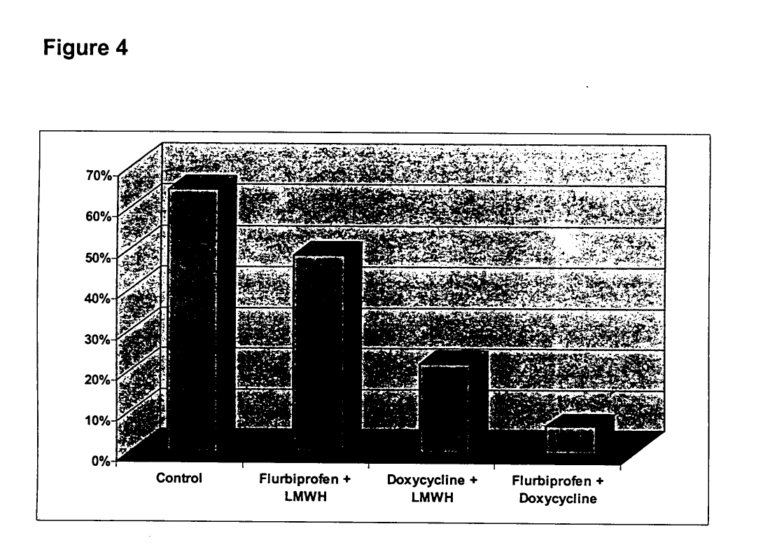

There was no statistically significant difference in the corneal scar size and burn intensity in any of the eyes. The mean percentage neovascularization for eyes administered flurbiprofen and low molecular weight heparin was 48.5±13.1. The mean percentage neovascularization for eyes administered flurbiprofen and doxycycline was 6.6±5.5. The mean percentage neovascularization for eyes administered doxycycline and low molecular weight heparin was 22.0±27.6. The mean percentage neovascularization for the control group was 64.6±9.9. Data are summarized in FIG. 4.

Neovascularization in each treatment group was statistically compared with the control and among the treatment groups using the Mann Whitney U analysis. Administration of flurbiprofen and doxycycline, and low molecular weight heparin and doxycycline, showed significantly lower corneal neovascularization when compared to the control group (p<0.05). Although administration of flurbiprofen and low molecular weight heparin showed a trend towards inhibition of corneal neovascularization when compared to the control, the inhibition was not significant (p=0.105).

When the groups were compared to each other, there was no significant difference between administration of low molecular weight heparin and doxycycline, nor was there a significant difference between administration of flurbiprofen and doxycycline (p=0.355). Similarly there was no significant difference between administration of low molecular weight heparin and doxycycline, and administration of flurbiprofen and low molecular weight heparin (p=0.069). There was, however, a significant difference between administration of flurbiprofen and doxycycline, and administration of flurbiprofen and low molecular weight heparin (p=0.02).

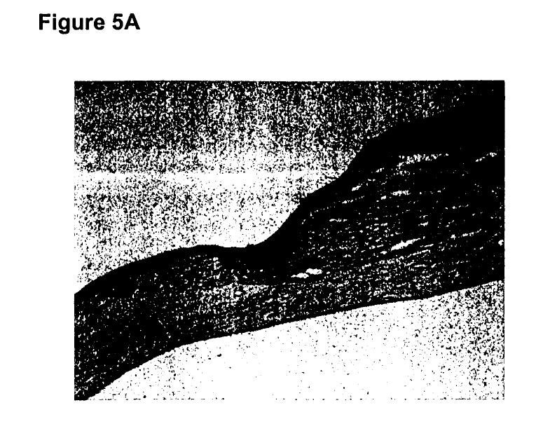

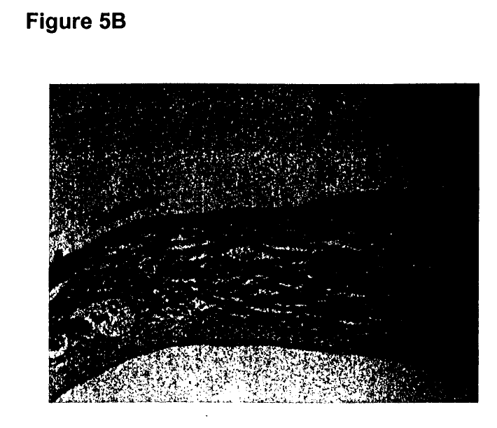

Histological preparations of eyes from each of the treatment groups were stained with hematoxylin and eosin and examined using light microscopy. The results are shown in FIG. 5. FIG. 5A is an eye administered flurbiprofen and doxycycline; there were no vessels in the central stroma. FIG. 5B is an eye administered normal saline; extensive neovascularization involved the central corneal stroma.

Light microscopy evaluation of the histological preparations from the different groups was consistent with the slit lamp evaluation. Although all the treatment groups had more of an anti-angiogenic action when compared to the control, the group to which flurbiprofen and doxycycline was administered had the greatest effect. This indicated that flurbiprofen and doxycycline provided the greatest inhibition of neovascularization among the groups evaluated.

Each of the three possible two drug combinations of flurbiprofen, doxycycline, and low molecular weight heparin were effective in inhibiting corneal neovascularization when compared to control. The combinations of doxycycline and low molecular weight heparin, and doxycycline and flurbiprofen, were more effective than the combination of flurbiprofen and low molecular weight heparin.

Flurbiprofen is a non-steroidal anti-inflammatory agent that inhibits the synthesis of prostaglandins. Prostaglandins are produced in corneal wound healing and angiogenesis. Thus, flurbiprofen suppresses actively proliferating corneal vessels.

Flurbiprofen (0.03%^{w/v}) and low molecular weight heparin (10 mg/ml), administered as individual agents, did not significantly decrease corneal neovascularization in this model (data not shown). Doxycycline did significantly inhibit (p<0.05%) corneal neovascularization when administered at 20 mg/ml and not when administered at 10 mg/ml.

As previously described, combinations of flurbiprofen, low molecular weight heparin and doxycycline were more effective than when these agents are used individually at similar or higher concentrations. Without being bound by a particular theory, a mechanism may be that each agent has a different mode/site of action in the angiogenesis process. The combination may decrease the individual side-effects of the agents and target angiogenesis at different steps. This may decrease the neovascularization response and avoid use of higher concentrations of potentially therapeutic agents with ocular side effects.

### Example 4

Forty eyes belonging to forty male Long Evans pigmented rats weighing 200 to 250 g were divided into different groups for this study. All of the procedures involving animals were conducted in accordance with the Association for Research in Vision and Ophthalmology resolution on the use of animals in research. The studies were approved by the Institutional Animal Care and Use Committee of Tulane University Health Sciences Center.

Prior to all procedures, the rats were anesthetized by using intraperitoneal injection of ketamine hydrochloride (25 mg/kg) with xylazine hydrochloride (5 mg/kg). After using proparacaine hydrochloride as a topical anaesthetic agent one cornea of each animal was cauterized by pressing an applicator stick (with a diameter of 1.8 mm) coated with 75% silver nitrate/25% potassium nitrate (Arzol Chemical Co., Keen, N.H.) to the central cornea for 10 seconds. To increase the reproducibility of the injuries, one investigator cauterized all animals. Excess silver nitrate was removed by rinsing the eyes with 5 ml of balanced salt solution and then gently blotting the eyes with tissue paper.

In the first group (n=7) topical normal saline was used to ensure that chemical burns were of sufficient depth and degree to result the desired neovascular response and to compare the results of other groups with them. Group two (n=6) was treated with topical ascomycin (A. G. Scientific, Inc., San Diego, Calif.) solution made by dilution to the concentration of 50 μg/ml. In group three (n=6) flurbiprofen sodium ophthalmic solution (0.03%) (Allergan, Irvine, Calif.) was used. In group four (n=7) doxycycline solution with the concentration of 20 mg/ml made by dilution of doxycycline vials (American Pharmaceutical Partners, Schaumburg, Ill.) was instilled topically. Group five (n=7) was also treated with topical instillation of low molecular weight heparin solution (Enoxaparin sodium injection, Aventis Pharmaceuticals Inc., Bridgewater, N.J.) diluted to 10 mg/ml. The last group (n=7) received topical instillation of triamcinolone acetonide (4 mg/ml) (Bristol-Myers Squibb Company, Princeton, N.J.).

Those treatments were applied immediately after cauterization in eyes in each group. Treatment (topical) was continued two times daily at equal intervals for 7 days. An evaluation of corneal burn intensity such as described by Mahoney was made by observing the amount of elevation above corneal surface and if there was no elevation the animal was excluded. Extent of the scar was also evaluated by calculating the percentage of corneal surface occupied by the scar.

Drops were applied a few seconds apart allowing the animals to blink between drops. Corneal photographs were taken at the slit-lamp microscope (SL-7E, Topcon, Tokyo Japan) under general anaesthesia on the 7^{th} day. Inhaled carbon dioxide was then used to sacrifice the rats while under deep anaesthesia. Cauterized eyes were enucleated and fixed in formaldehyde 10% for one week. Corneal sections were prepared from all the eyes and histological exam using H&E staining was performed.

The colour slides of the cornea were converted to digital images using a scanner (Cano scan 9900F, Canon, Tokyo, Japan). The area of each cornea and its neovascularization was measured separately by using image j software (Wayne Rasband at the Research Services Branch, National Institute of Mental Health, Bethesda, Md.) and percentage of cornea occupied by vessels and corneal scar was calculated separately. A drawing of corneal blood vessels was made by one of investigators to compare with digital photos and to be sure that no vascular area is missing during calculation of percent area. Statistical analyses of neovascular and scar percent area in each group were performed using a General Linear Models (GLM) procedure with a Tukey's studentized range test which controls the Type I experimentwise error rate (SAS version 8,02 Cary, N.C.). Statistical significance was set at p≦0.05.

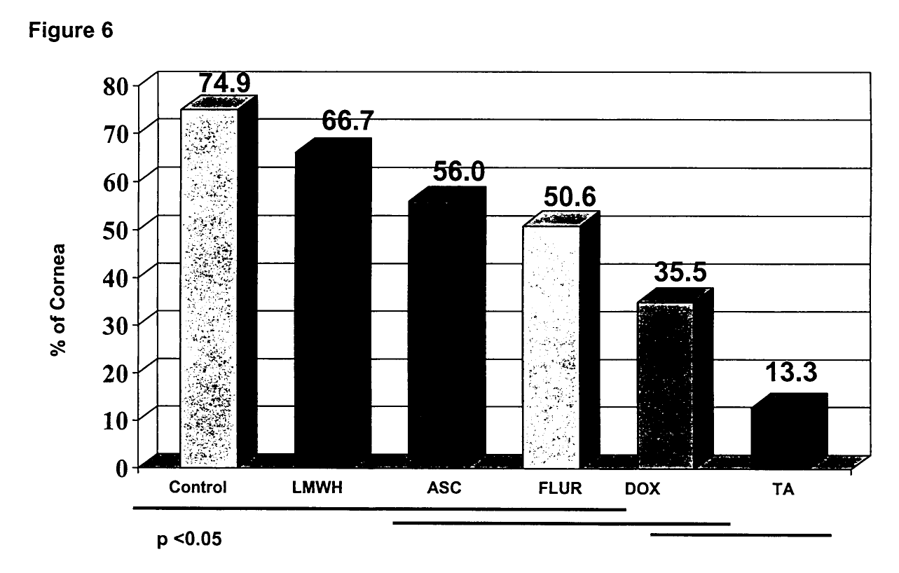

The percentage of burn scar area and neovascularization (relative to total corneal area) in each animal is shown in Table 1. The mean of percent area in the control group was 74.9%±9.2%, while it was 66.7%±9.9%, 56.0%±22.4%, 50.5%±18.7%, 35.5%±29.1%, and 13.3%±7.1% respectively in the LMWH, ascomycin flurbiprofen, doxycycline, and triamcinolone groups (FIG. 6).

TABLE 1
Percent area of neovascularization and percent area
of scar in each cornea of different animal groups.

| | Percent Area Of | Percent Area Of |
|---|---|---|
| | Neovascularization | Scar |
| Agent Used | Mean +/− SD | Mean +/− SD |
| Normal saline | 74.9 +/− 9.2 | 17.3 +/− 3.5 |
| LMWH | 66.7 +/− 9.9 | 15.5 +/− 2.2 |
| Ascomycin | 56.0 +/− 22.4 | 16.7 +/− 8.2 |
| Flurbiprofen | 50.6 +/− 18.7 | 18.8 +/− 5.2 |
| Doxycycline | 35.5 +/− 29.1 | 16.7 +/− 2.8 |
| Triamcinolone | 13.3 +/− 7.2 | 17.3 +/− 2.6 |

There were no statistically significant differences in NV area among the control group and the LMWH, ascomycin and flurbiprofen groups. There were also no significant differences among the ascomycin, flurbiprofen and doxycycline groups or between the doxycycline and triamcinolone groups. There was a significant reduction in NV area in the doxycycline and triamcinolone groups compared to the control group and the LMWH group and the triamcinolone group also demonstrated a significant reduction in NV area compared to ascomycin and flurbiprofen groups.

There was no significant difference in percentage of burn scar area between the control and any of the study groups.

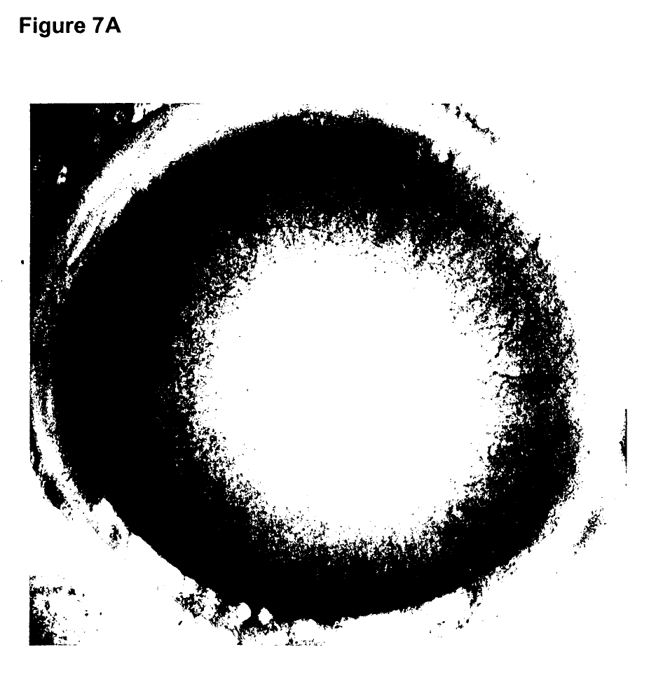

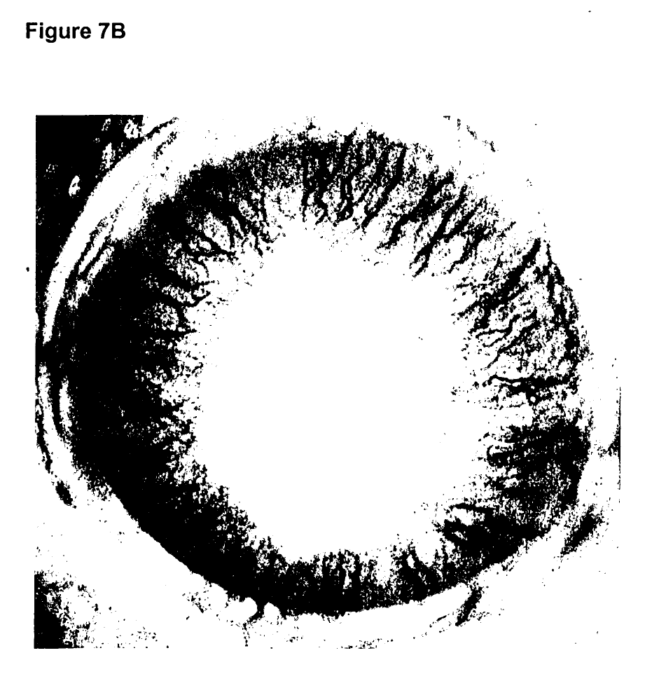

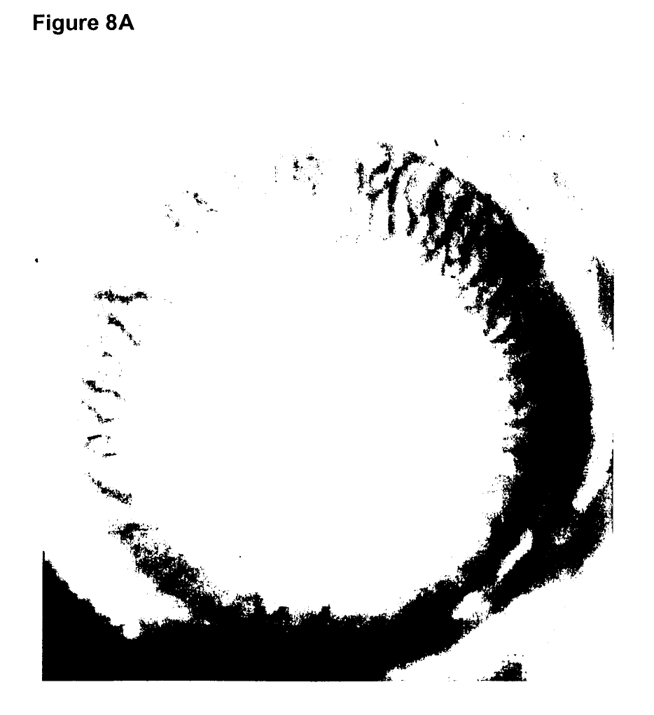

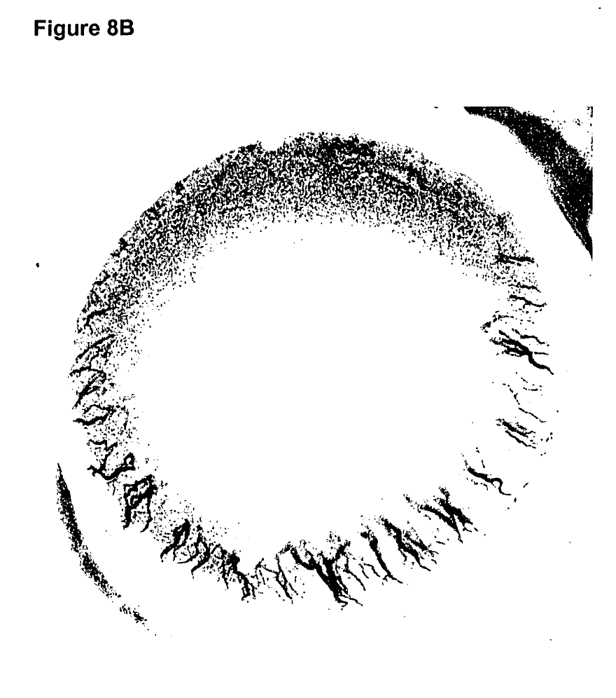

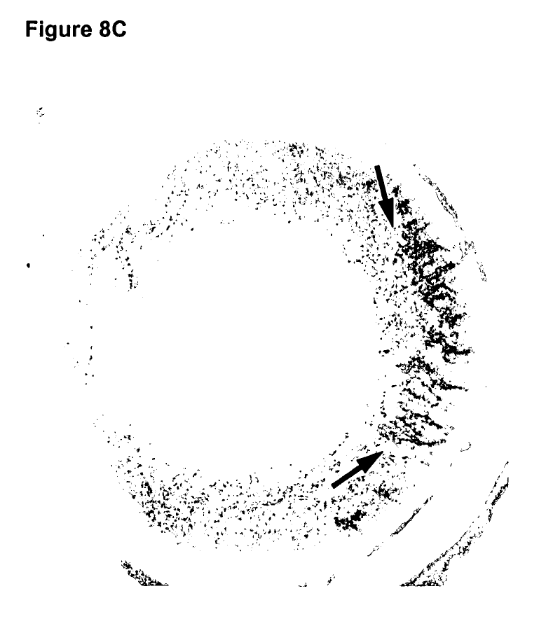

A representative corneal picture in the control group is shown in FIGS. 7A and B. FIG. 8A is a digitally enhanced slit lamp photograph of the cornea seven days after induction of corneal burn in eyes treated with flurbiprofen (neovascularization is quite prominent in this group). FIG. 8B is a digitally enhanced slit lamp photograph of the cornea seven days after induction of corneal burn in eyes treated with doxycycline (neovascularization is less prominent than in control group). FIG. 8C is a digitally enhanced slit lamp photograph of the cornea seven days after induction of corneal burn in eyes treated with triamcinolone acetonide (arrows circumscribe the relatively small neovascular area).

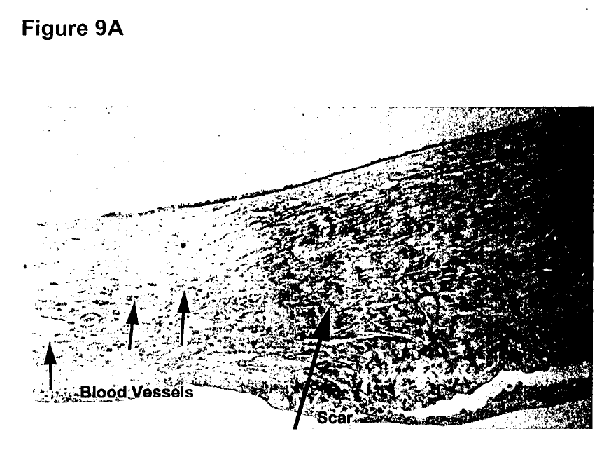

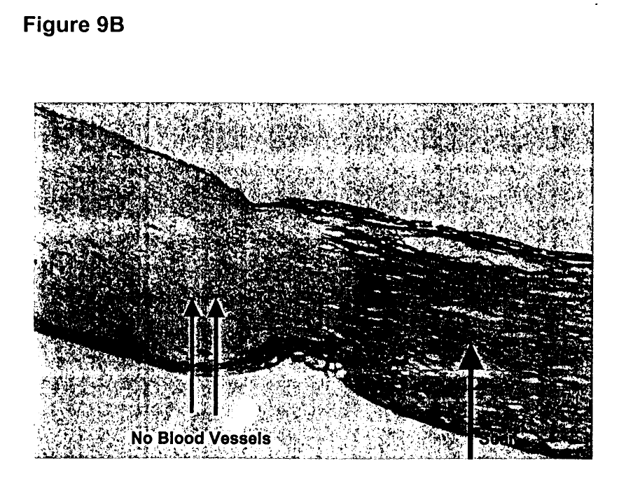

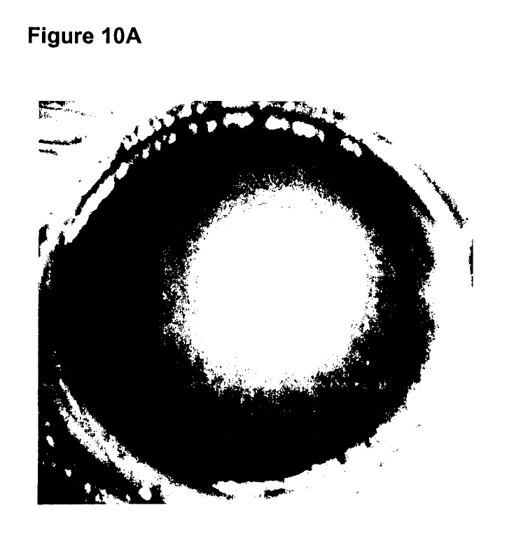

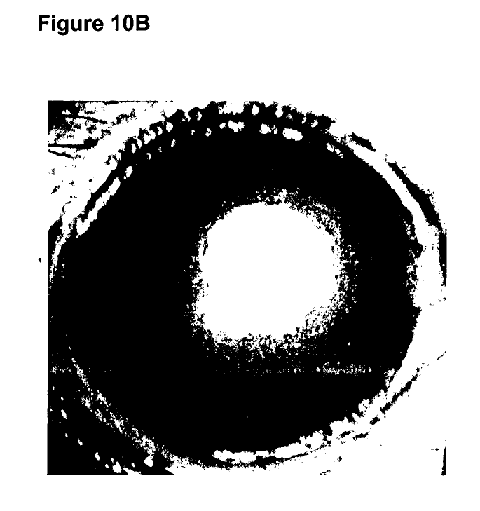

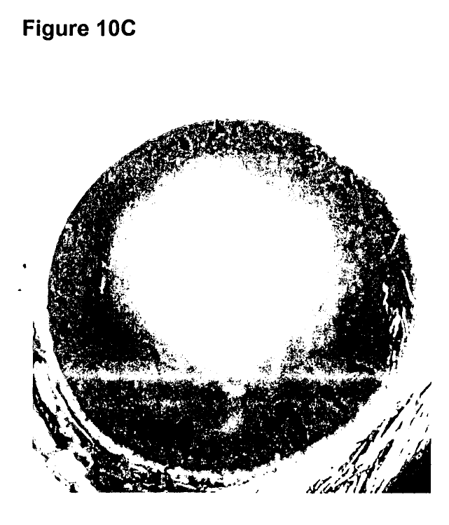

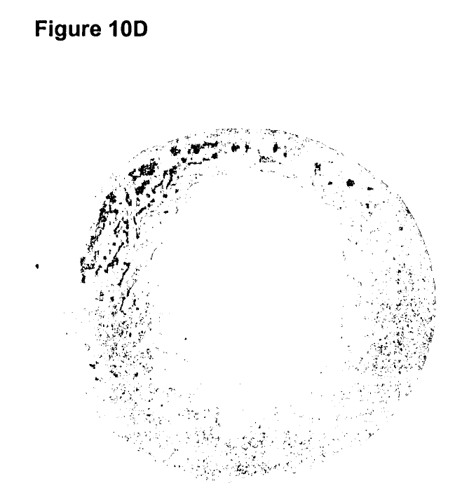

Representative histologic sections of the control and triamcinolone groups are shown in FIG. 9. FIG. 9A is a photograph of a histopathology preparation of the corneal burn in a control eye treated with normal saline, showing corneal scar (large arrow) and new vessels (small arrows) in the corneal stroma. H&E 100X. FIG. 9B is a photograph of a histopathology preparation of the corneal burn in an eye treated with triamcinolone acetonide (double arrows point to avascular stroma). Note extensive neovascularization of the corneal stroma in FIG. 10A compared to FIG. 10B. H&E 100X.

### Example 5

Twenty-four eyes of 24 Male Long Evans pigmented rats weighing 200 g to 250 g were divided into 3 different groups for this study. All of the procedures involving animals were conducted in accordance with the Association for Research in Vision and Ophthalmology resolution on the use of animals in research. All animals were housed in individual cages and maintained under standard conditions. The Institutional Animal Care and Use Committee of Tulane University Health Sciences Center approved the experimental protocol.

To induce corneal neovascularization in rats, a silver nitrate cauterization technique described by Mahoney et al [Drug effects on the neovascularization response to silver nitrate cauterization of the rat cornea Curr Eye Res 1985; 4:531-35] was used. All procedures were performed under general anesthesia induced by intraperitoneally administered ketamine hydrochloride and xylazine combination (94.7 mg/kg body weight). After applying 0.5% proparacaine hydrochloride as a topical anaesthetic agent one cornea of each animal was cauterized by pressing an applicator stick (with a diameter of 1.8 mm) coated with 75% silver nitrate/25% potassium nitrate (Arzol chemical co., Keen, N.H.) to the central cornea for 10 seconds (timed using a stopwatch) under the operating microscope. Excess silver nitrate was removed by rinsing the eyes with 5 ml of a balanced salt solution and then gently blotting the eyes with tissue paper. To increase the reproducibility of the injuries, one investigator cauterized all animals. Following cauterization, the rats were randomized into drug groups to eliminate any potential bias in the degree of burns between groups. Two drops of each drug were applied topically to each cornea immediately following cauterization.

The rats were divided into three groups. Group 1 (n=8) received 4 mg/ml triamcinolone acetonide (Kenalog-40; Bristol-Myers Squibb Company, Princeton, N.J.) and 10 mg/ml low molecular heparin (Enoxaparin: Aventis Pharmaceuticals Inc., Bridgewater, N.J.), Group 2 (n=8) 4 mg/ml triamcinolone acetonide and 20 mg/ml doxycycline (American Pharmaceutical Partners, Schaumburg, Ill.), and Group 3 (n=8) saline. All doses were topically administered as a single drop applied two times per day for 7 days. Treatment was started immediately after cauterization in all groups.

All animals were anesthetized as described above and their corneas evaluated by slit-lamp microscopy on the 3rd and 6th day. Corneal photographs were taken with x×25 magnification using a camera attached to the slit-lamp microscope (Topcon SL-7E, Tokyo, Japan) on the 7th day. Neovascularization in each cornea was evaluated using the technique described by Mahoney et al [Drug effects on the neovascularization response to silver nitrate cauterization of the rat cornea Curr Eye Res 1985; 4:531-35] by an examiner who was masked with regard to the treatment groups to minimize observer bias. For each eye, the extent of burn stimulus response was scored as; 0 (no blister, not raised above corneal surface), +1 (small blister, raised slightly above the surface), +2 (medium blister, raised moderately above the surface), +3 (large blister). Only the corneas with an initial burn stimulus score of +2 or higher were included for the calculation of the mean burn stimulus and neovascularization scores in each group. All photographs were converted to high resolution digital forms by scanner (Cano scan 9900F, Canon, Tokyo, Japan). The corneal surface covered with neovascular vessels was measured on the photographs as the percentage of the total area of the cornea. The area of each cornea and its neovascularization was measured separately by using image j software (Wayne Rasband at the Research Services Branch, National Institute of Mental Health, Bethesda, Md.) and percentage of cornea occupied by vessels and corneal scar was calculated separately.

The area of neovascularization was measured and its ratio to the entire corneal area was determined as the percentage of corneal neovascularization. A drawing of corneal blood vessels was made by one of investigators to compare with digital photos and to be sure that no vascular area was missing during calculation of percent area. In addition, extent of the scar was also evaluated by calculating the percentage of corneal surface covered by the scar.

Percent inhibition was calculated by comparing the mean percentage of neovascularization in each drug-treated group to that in the control group. After scoring the burn stimulus and the percentage of neovascularization for all groups, the animals were sacrificed on the seventh day.

Statistical analyses of neovascular and scar percent area in each group were performed using a General Linear Models (GLM) procedure with a Tukey's studentized range test, which controls the Type I experiment wise error rate (SAS version 8,02 Cary, N.C.). Statistical significance was set at p<0.01.

Following sedation using the intraperitoneally administered ketamine hydrochloride and xylazine combination (94.7 mg/kg body weight), enucleation was performed before the animals were euthanized. Immediately after enucleation, the globes were penetrated with a 27-gauge needle, 1.0 mm from the limbus at the 3 and 9 o'clock meridians to allow the fixative to fill the eyes rapidly. The eyes were prepared for histologic examination using 10% formaldehyde. After fixation for 24 hours, the eyes were removed from the fixative and corneas were dehydrated and sectioned. The corneas are then soaked in xylene and paraffin, later they were embedded in paraffin and cut at 1 μm for staining with hematoxilin-eosin (H&E) for light microscopy.

Light microscopic examination was performed on every microscopic section. Sections were examined by dividing the corneas into two halves through the center of the lesion and were evaluated with regard to the intensity of new vessels, polymorphonuclear (PMN) leukocytes, edema, and fibroblastic activity.

The burn stimulus and percentage of neovascularization (relative to total corneal area) and histopathologic scores of each cornea in the treatment and placebo groups are shown in Table 2.

TABLE 2

| | | |
|---|---|---|
| | Percent area of | Burn stimulus |
| Drug/Animal No | Neovascularization | Score |

TA and LMWH

| | | |
|---|---|---|
| 1 | 14 | 3 |
| 2 | 49.3 | 3 |
| 3 | 40.6 | 3 |
| 4 | 12.2 | 3 |
| 5 | 3.1 | 3 |
| 6 | 27.1 | 3 |
| 7 | 2 | 2 |
| 8 | 0 | 3 |

TA and Dx

| | | |
|---|---|---|
| 1 | 9 | 3 |
| 2 | 29 | 3 |
| 3 | 0 | 2 |
| 4 | 0 | 3 |
| 5 | 0 | 3 |
| 6 | 0 | 3 |
| 7 | 8.4 | 3 |
| 8 | 0 | 3 |

Control

| | | |
|---|---|---|
| 1 | 64.8 | 3 |
| 2 | 74.4 | 3 |
| 3 | 65.8 | 3 |
| 4 | 49.3 | 3 |
| 5 | 51.6 | 2 |
| 6 | 78.6 | 3 |
| 7 | 67.7 | 3 |
| 8 | 65.4 | 3 |

TA: Triamcinolone acetonide,
LMWH: Low molecular weight heparin,
Dx: Doxycycline

The burn stimulus score was +2 or higher in all eyes. The mean burn stimulus score was not statistically significantly different between the treatment and the placebo groups (p=1.0). On slit lamp examination, all eyes treated with the combination of triamcinolone and low molecular heparin, or the combination of triamcinolone with doxycycline showed less inflammation during the treatment period with less eyelid edema and less ciliary injection compared to the control eyes.

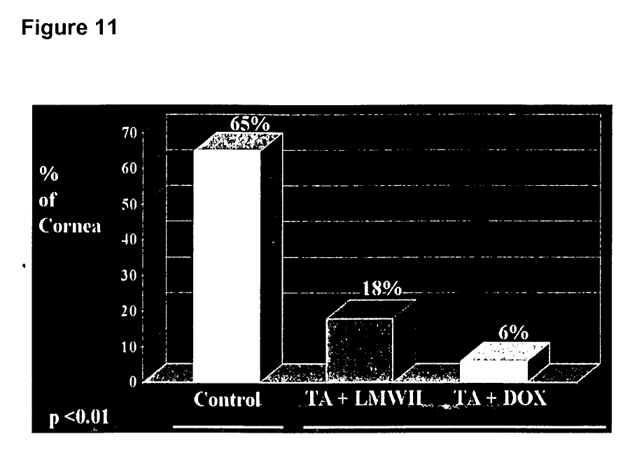

Representative slit lamp photographs of the corneas of the 3 groups are shown in FIG. 10. The means percent area of corneal neovascularization in combination of triamcinolone with LMWH; the combination of triamcinolone with doxycycline, and control groups were 18.5±18.6%, 5.8±10.1%, 64.7±10.0%, respectively (FIG. 11). The mean percent area of neovascularization in triamcinolone with LMWH or triamcinolone with doxycycline groups was significantly different from control group (P<0.001, for both). There was no significant difference between study groups.

There was no significant difference in percent area of corneal scar between different groups (P>0.05).

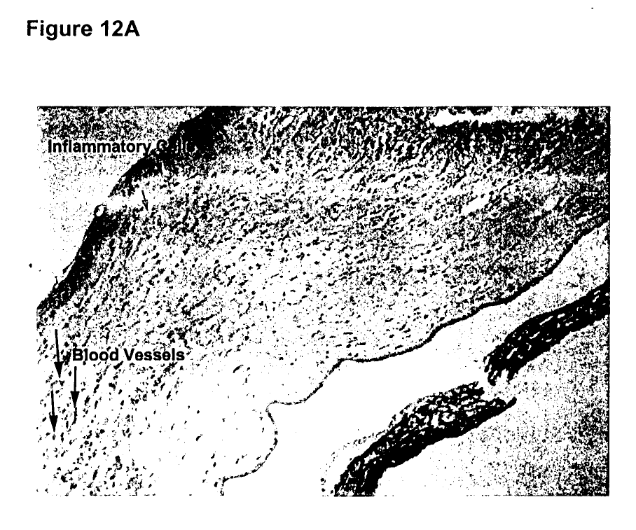

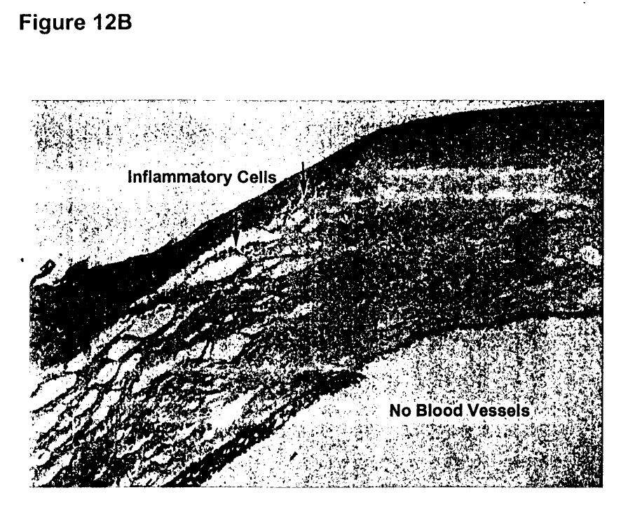

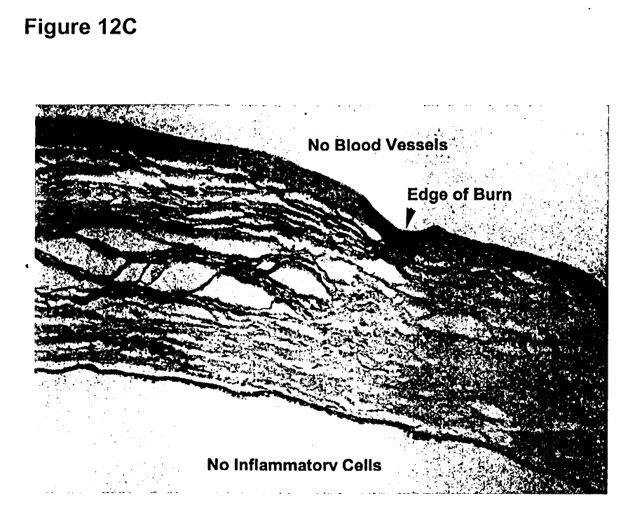

Histological evaluation of the corneas showed corneal neovascularization and inflammation in the control group (FIG. 12A). The corneas of the triamcinolone and LMWH showed decreased corneal neovascularization with minimal inflammatory response (FIG. 12B). There was almost no neovascularization with trace inflammatory response in the triamcinolone and doxycycline group (FIG. 12C).

The invention is further described by the following numbered paragraphs:
1. A formulation for the treatment of ocular neovascularization comprising heparin in a concentration and dose suitable for treating ocular neovascularization, characterized in that said compound is in a pharmaceutically acceptable form suitable for delivery to the eye.
2. A formulation for the treatment of ocular neovascularization comprising heparin in a concentration and dose suitable for treating ocular neovascularization, characterized in that said compound is at a substantially neutral pH in a pharmaceutically acceptable form suitable for delivery to the eye.
3. An ocular pharmaceutically acceptable formulation according to paragraph 1 wherein the heparin is low molecular weight heparin.
4. An ocular pharmaceutically acceptable formulation according to paragraph 1 wherein the heparin is in a concentration from about 0.01 pg/ml to about 100 mg/ml.
5. A formulation for the treatment of ocular neovascularization according to paragraph 1 wherein formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization
6. A formulation for the treatment of ocular neovascularization according to paragraph 5 wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3).
7. A formulation for the treatment of ocular neovascularization according to paragraph 6 wherein doxycycline is the tetracycline derivative employed in the formulation.
8. A formulation for the treatment of ocular neovascularization according to paragraph 1 wherein the formulation also includes a steroid at a concentration from about 0.1 mg/ml to about 40 mg/ml.
9. A formulation according to paragraph 8 wherein the steroid is selected from the group consisting of: triamcinolone, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate.
10. A formulation according to paragraph 9 wherein the steroid is 9-fluoro-11,21-ihydroxy-16,17-[1-methylethylidinebis(oxy)]pregna-1,4-diene-3,20-dione.
11. A formulation for the treatment of ocular neovascularization according to paragraph 1 wherein the formulation also includes an anti-prostaglandin in a concentration from about 1 μg/ml to about 10 mg/ml.
12. A formulation according to paragraph 11 wherein the anti-prostaglandin is selected from the group consisting of: flurbiprofen, indomethacin, ketorolac, tromethamine, meclofenamate, flurbiprofen, and compounds in the pyrrolo-pyrrole group of non-steroidal anti-inflammatory drugs.
13. A formulation according to paragraph 12 wherein the anti-prostaglandin is flurbiprofen.
14. A formulation for the treatment of ocular neovascularization according to paragraph 1 wherein the formulation also includes an antimicrobial in a concentration from about 20 μg/ml to about 200 μg/ml.
15. A formulation according to paragraph 14 wherein the antimicrobial is a macrolide antibiotic.
16. A formulation according to paragraph 15 wherein the macrolide antibiotic is selected from the group consisting of: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.
17. A formulation according to paragraph 15 wherein the macrolide antibiotic is ascomycin.
18. A formulation for the treatment of ocular neovascularization according to paragraph 1 wherein the formulation also includes an inhibitor of a metalloproteinase in a concentration and dose to reduce ocular neovascularization.
19. A formulation according to paragraph 18 wherein the inhibitor of a metalloproteinase is selected from the group consisting of: naturally occurring proteins such as TIMP-1 that specifically inhibit matrix metalloproteinases, and synthetic metalloproteinase inhibitors including Batimastat (BB-94) and marimastat (BB-2516), collagenase I and III (MMP-1 and MMP-13), gelatinase A and B (MMP-2 and -9), stromelysin (MMP-3), matrilysin (MMP-7) or membrane type MMP (MMP-14).
20. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml;
21. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an anti-prostaglandin such as flurbiprofen at a concentration from about 1 μg/ml to about 10 mg/ml;
22. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an macrolide antibiotic such as ascomycin at a concentration from about 20 μg/ml to about 200 μg/ml;
23. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an anti-prostaglandin such as flurbiprofen at a concentration from about 1 μg/ml to about 10 mg/ml and an macrolide antibiotic such as ascomycin at a concentration from about 20 μg/ml to about 200 μg/ml;
24. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and an anti-prostaglandin such as flurbiprofen at a concentration from about 1 μg/ml to about 10 mg/ml and an inhibitor of a metalloproteinase in a concentration and dose to reduce ocular neovascularization;
25. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a steroid such as triamcinolone acetonide at a concentration from about 0.1 mg/ml to about 40 mg/ml and an inhibitor of a metalloproteinase in a concentration and dose to reduce ocular neovascularization;
26. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml; or
27. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an macrolide antibiotic such as ascomycin at a concentration from about 20 μg/ml to about 200 μg/ml; or
28. An ocular pharmaceutically acceptable formulation comprising: (a) a heparin or low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization and a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml and an macrolide antibiotic such as ascomycin at a concentration from about 20 μg/ml to about 200 μg/ml.
29. A method for treating ocular neovascularization comprising the step of: administering to a patient a low molecular weight heparin in a concentration from about 0.01 pg/ml to about 100 mg/ml, characterized in that said compound is at a substantially neutral pH in a pharmaceutically acceptable form suitable for delivery to the eye to reduce ocular neovascularization.
30. A method for treating ocular neovascularization comprising the step of: administering to a patient a formulation as defined in paragraphs 1 to 28 for sufficient time to treat the ocular neovascularization.
31. A method for reducing ocular irritation comprising the step of: administering to a patient a formulation as defined in anyone of paragraph 1 to 28 following corneal surgery.
32. A method for treating ocular neovascularization comprising the step of: administering one or more of the formulations defined in paragraphs 1 to 28 to a patient in a cyclic tumor treatment regimen to reduce blood vessel growth and proliferation at a tumor site.
33. A method according to anyone of paragraphs 29 to 32 which also includes the step of administering an anti-angiogenic agent designed to block the actions of VEGF on endothelial cells.
34. A method according to anyone of paragraphs 33 wherein the anti-angiogenic agent is a rhuFab V2 or a humanized AMD-Fab or an anti-VEGF aptamer.
35. An ocular pharmaceutically acceptable formulation according to paragraph 1 and as substantially as herein described.
36. A method according to paragraph 29 and as substantially as herein described.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention, as defined by the following claims.

What is claimed is:

1. A formulation for treating ocular neovascularization comprising a composition comprising heparin in a concentration and dose suitable for treating ocular neovascularization, characterized in that said composition is in a pharmaceutically acceptable form suitable for delivery to the eye.

2. The formulation of claim 1, wherein said composition is at a substantially neutral pH or
wherein the heparin is low molecular weight heparin or
wherein the heparin is in a concentration from about 0.01 pg/ml to about 100 mg/ml or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) and
wherein doxycycline is the tetracycline derivative employed in the formulation or
wherein the formulation also includes a steroid at a concentration from about 0.1 mg/ml to about 40 mg/ml or
wherein the formulation also includes a steroid at a concentration from about 0.1 mg/ml to about 40 mg/ml and
wherein the steroid is selected from the group consisting of: triamcinolone, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate or
wherein the formulation also includes a steroid at a concentration from about 0.1 mg/ml to about 40 mg/ml and
wherein the steroid is 9-fluoro-11,21-ihydroxy-16,17-[1-methylethylidinebis(oxy)]pregna-1,4-diene-3,20-dione or
wherein the formulation also includes an anti-prostaglandin in a concentration from about 1 μg/ml to about 10 mg/ml or
wherein the formulation also includes an anti-prostaglandin in a concentration from about 1 μg/ml to about 10 mg/ml and
wherein the anti-prostaglandin is selected from the group consisting of: flurbiprofen, indomethacin, ketorolac, tromethamine, meclofenamate, flurbiprofen, and compounds in the pyrrolo-pyrrole group of non-steroidal anti-inflammatory drugs or
wherein the formulation also includes an anti-prostaglandin in a concentration from about 1 μg/ml to about 10 mg/ml and
wherein the anti-prostaglandin is flurbiprofen or
wherein the formulation also includes an antimicrobial in a concentration from about 20 μg/ml to about 200 μg/ml or
wherein the formulation also includes an antimicrobial in a concentration from about 20 μg/ml to about 200 μg/ml and
wherein the antimicrobial is a macrolide antibiotic or
wherein the formulation also includes an antimicrobial in a concentration from about 20 μg/ml to about 200 μg/ml and
wherein the antimicrobial is a macrolide antibiotic and
wherein the macrolide antibiotic is selected from the group consisting of: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide or
wherein the formulation also includes an antimicrobial in a concentration from about 20 μg/ml to about 200 μg/ml and
wherein the antimicrobial is a macrolide antibiotic and
wherein the macrolide antibiotic is ascomycin or
wherein the formulation also includes an inhibitor of a metalloproteinase in a concentration and dose to reduce ocular neovascularization or
wherein the formulation also includes an inhibitor of a metalloproteinase in a concentration and dose to reduce ocular neovascularization and
wherein the inhibitor of a metalloproteinase is selected from the group consisting of: naturally occurring proteins such as TIMP-1 that specifically inhibit matrix metalloproteinases, and synthetic metalloproteinase inhibitors including Batimastat (BB-94) and marimastat (BB-2516), collagenase I and III (MMP-1 and MMP-13), gelatinase A and B (MMP-2 and -9), stromelysin (MMP-3), matrilysin (MMP-7) or membrane type MMP (MMP-14).

3. An ocular pharmaceutically acceptable formulation comprising a composition comprising:
(a) a heparin or low molecular weight heparin in a concentration from about 0.01 pg/ml to about 100 mg/ml, characterized in that said composition is at a substantially neutral pH in a pharmaceutically acceptable form suitable for delivery to the eye to reduce ocular neovascularization and
(b) a steroid, an anti-prostoglandin, an inhibitor of metalloproteinase, a tetracycline, a macrolide antibiotic or any combination thereof.

4. The formulation of claim 3 wherein (b) is a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml or
wherein (b) is a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is an anti-prostaglandin such as flurbiprofen at a concentration from about 1 μg/ml to about 10 mg/ml and an macrolide antibiotic such as ascomycin at a concentration from about 20 μg/ml to about 200 μg/ml or
wherein (b) is an anti-prostagiandin such as flurbiprofen at a concentration from about 1 μg/ml to about 10 mg/ml and an inhibitor of a metalloproteinase in a concentration and dose to reduce ocular neovascularization or
wherein (b) is a steroid such as triamcinolone acetonide at a concentration from about 0.1 mg/ml to about 40 mg/ml and an inhibitor of a metalloproteinase in a concentration and dose to reduce ocular neovascularization or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml and an macrolide antibiotic such as ascomycin at a concentration from about 20 μg/ml to about 200 μg/ml.

5. A method for treating ocular neovascularization comprising administering to a patient a formulation comprising a composition comprising heparin in a concentration and dose suitable for treating ocular neovascularization, wherein said composition is in a pharmaceutically acceptable form suitable for delivery to the eye, for a sufficient time to treat the ocular neovascularization.

6. The method of claim 5, wherein the formulation comprises a low molecular weight heparin in a concentration from about 0.01 pg/ml to about 100 mg/ml, characterized in that said compound is at a substantially neutral pH in a pharmaceutically acceptable form suitable for delivery to the eye.

7. The method of claim 5, wherein the formulation comprises a composition comprising heparin in a concentration and dose suitable for treating ocular neovascularization, characterized in that said composition is in a pharmaceutically acceptable form suitable for delivery to the eye or
wherein said composition is at a substantially neutral pH or
wherein the heparin is low molecular weight heparin or
wherein the heparin is in a concentration from about 0.01 pg/ml to about 100 mg/ml or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) and
wherein doxycycline is the tetracycline derivative employed in the formulation or
wherein the formulation also includes a steroid at a concentration from about 0.1 mg/ml to about 40 mg/ml or
wherein the formulation also includes a steroid at a concentration from about 0.1 mg/ml to about 40 mg/ml and
wherein the steroid is selected from the group consisting of: triamcinolone, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate or
wherein the formulation also includes a steroid at a concentration from about 0.1 mg/ml to about 40 mg/ml and
wherein the steroid is 9-fluoro-11,21-ihydroxy-16,17-[1-methylethylidinebis(oxy)]pregna-1,4-diene-3,20-dione or
wherein the formulation also includes an anti-prostaglandin in a concentration from about 1 μg/ml to about 10 mg/ml or
wherein the formulation also includes an anti-prostaglandin in a concentration from about 1 μg/ml to about 10 mg/ml and
wherein the anti-prostaglandin is selected from the group consisting of: flurbiprofen, indomethacin, ketorolac, tromethamine, meclofenamate, flurbiprofen, and compounds in the pyrrolo-pyrrole group of non-steroidal anti-inflammatory drugs or
wherein the formulation also includes an anti-prostaglandin in a concentration from about 1 μg/ml to about 10 mg/ml and
wherein the anti-prostaglandin is flurbiprofen or
wherein the formulation also includes an antimicrobial in a concentration from about 20 μg/ml to about 200 μg/ml or
wherein the formulation also includes an antimicrobial in a concentration from about 20 μg/ml to about 200 μg/ml and
wherein the antimicrobial is a macrolide antibiotic or
wherein the formulation also includes an antimicrobial in a concentration from about 20 μg/ml to about 200 μg/ml and
wherein the antimicrobial is a macrolide antibiotic and
wherein the macrolide antibiotic is selected from the group consisting of: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide or
wherein the formulation also includes an antimicrobial in a concentration from about 20 μg/ml to about 200 μg/ml and
wherein the antimicrobial is a macrolide antibiotic and
wherein the macrolide antibiotic is ascomycin or
wherein the formulation also includes an inhibitor of a metalloproteinase in a concentration and dose to reduce ocular neovascularization or
wherein the formulation also includes an inhibitor of a metalloproteinase in a concentration and dose to reduce ocular neovascularization and
wherein the inhibitor of a metalloproteinase is selected from the group consisting of: naturally occurring proteins such as TIMP-1 that specifically inhibit matrix metalloproteinases, and synthetic metalloproteinase inhibitors including Batimastat (BB-94) and marimastat (BB-2516), collagenase I and III (MMP-1 and MMP-13), gelatinase A and B (MMP-2 and -9), stromelysin (MMP-3), matrilysin (MMP-7) or membrane type MMP (MMP-14).

8. The method of claim 5, wherein the formulation comprises a composition comprising:
(a) a heparin or low molecular weight heparin in a concentration from about 0.01 pg/ml to about 100 mg/ml, characterized in that said composition is at a substantially neutral pH in a pharmaceutically acceptable form suitable for delivery to the eye to reduce ocular neovascularization and
(b) a steroid, an anti-prostogiandin, an inhibitor of metalloproteinase, a tetracycline, a macrolide antibiotic or any combination thereof.

9. The method of claim 8, wherein (b) is a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml or
wherein (b) is a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is an anti-prostaglandin such as flurbiprofen at a concentration from about 1 μg/ml to about 10 mg/ml and an macrolide antibiotic such as ascomycin at a concentration from about 20 μg/ml to about 200 μg/ml or
wherein (b) is an anti-prostaglandin such as flurbiprofen at a concentration from about 1 μg/ml to about 10 mg/ml and an inhibitor of a metalloproteinase in a concentration and dose to reduce ocular neovascularization or
wherein (b) is a steroid such as triamcinolone acetonide at a concentration from about 0.1 mg/ml to about 40 mg/ml and an inhibitor of a metalloproteinase in a concentration and dose to reduce ocular neovascularization or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml and an macrolide antibiotic such as ascomycin at a concentration from about 20 μg/ml to about 200 μg/ml.

10. The method of claim 5, wherein the method further comprises administering the formulation to a patient in a cyclic tumor treatment regimen to reduce blood vessel growth and proliferation at a tumor site or
wherein the method further comprises the step of administering an anti-angiogenic agent designed to block the actions of VEGF on endothelial cells or
wherein the method further comprises the step of administering an anti-angiogenic agent designed to block the actions of VEGF on endothelial cells and
wherein the anti-angiogenic agent is a rhuFab V2 or a humanized AMD-Fab or an anti-VEGF aptamer.

11. A method for reducing ocular irritation comprising administering to a patient a formulation comprising a composition comprising heparin in a concentration and dose suitable for treating ocular neovascularization, characterized in that said composition is in a pharmaceutically acceptable form suitable for delivery to the eye following corneal surgery.

12. The method of claim 11 wherein said composition is at a substantially neutral pH or
wherein the heparin is low molecular weight heparin or
wherein the heparin is in a concentration from about 0.01 pg/ml to about 100 mg/ml or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) or
wherein the formulation also includes a tetracycline or a derivative thereof including chemically modified tetracyclines which inhibit matrix metalloproteinase activity, characterized in that said tetracycline or derivative thereof is at in a pharmaceutically acceptable form suitable for delivery to the eye in an amount sufficient to reduce ocular neovascularization and
wherein the tetracycline or a derivative thereof is selected from the group consisting of: doxycycline, demeclocycline, minocycline, oxytetracycline, lymecycline, CMT-315, CMT-3, CMT-8, CMT-308 or 6-demethyl-6-deoxy-4-dedimethylamino tetracylcine (COL-3) and
wherein doxycycline is the tetracycline derivative employed in the formulation or
wherein the formulation also includes a steroid at a concentration from about 0.1 mg/ml to about 40 mg/ml or
wherein the formulation also includes a steroid at a concentration from about 0.1 mg/ml to about 40 mg/ml and
wherein the steroid is selected from the group consisting of: triamcinolone, budesonide, cortisone, dexamethasone, hydrocortisone, methylprednisolone, prednisolone, prednisone sodium phosphate, fluorometholone, fluorometholone alcohol, rimexolone, medrysone alcohol, lotoprednol etabonate, 11-desoxcortisol, and anecortave acetate or
wherein the formulation also includes a steroid at a concentration from about 0.1 mg/ml to about 40 mg/ml and
wherein the steroid is 9-fluoro-11,21-ihydroxy-16,17-[1-methylethylidinebis(oxy)]pregna-1,4-diene-3,20-dione or
wherein the formulation also includes an anti-prostaglandin in a concentration from about 1 μg/ml to about 10 mg/ml or
wherein the formulation also includes an anti-prostaglandin in a concentration from about 1 μg/ml to about 10 mg/ml and
wherein the anti-prostaglandin is selected from the group consisting of: flurbiprofen, indomethacin, ketorolac, tromethamine, meclofenamate, flurbiprofen, and compounds in the pyrrolo-pyrrole group of non-steroidal anti-inflammatory drugs or
wherein the formulation also includes an anti-prostaglandin in a concentration from about 1 μg/ml to about 10 mg/ml and
wherein the anti-prostaglandin is flurbiprofen or
wherein the formulation also includes an antimicrobial in a concentration from about 20 μg/ml to about 200 μg/ml or
wherein the formulation also includes an antimicrobial in a concentration from about 20 μg/ml to about 200 μg/ml and
wherein the antimicrobial is a macrolide antibiotic or
wherein the formulation also includes an antimicrobial in a concentration from about 20 μg/ml to about 200 μg/ml and
wherein the antimicrobial is a macrolide antibiotic and
wherein the macrolide antibiotic is selected from the group consisting of: tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide or
wherein the formulation also includes an antimicrobial in a concentration from about 20 μg/ml to about 200 μg/ml and
wherein the antimicrobial is a macrolide antibiotic and
wherein the macrolide antibiotic is ascomycin or
wherein the formulation also includes an inhibitor of a metalloproteinase in a concentration and dose to reduce ocular neovascularization or
wherein the formulation also includes an inhibitor of a metalloproteinase in a concentration and dose to reduce ocular neovascularization and
wherein the inhibitor of a metalloproteinase is selected from the group consisting of: naturally occurring proteins such as TIMP-1 that specifically inhibit matrix metalloproteinases, and synthetic metalloproteinase inhibitors including Batimastat (BB-94) and marimastat (BB-2516), collagenase I and III (MMP-1 and MMP-13), gelatinase A and B (MMP-2 and -9), stromelysin (MMP-3), matrilysin (MMP-7) or membrane type MMP (MMP-14).

13. The method of claim 11, wherein the formulation comprises a composition comprising:
(a) a heparin or low molecular weight heparin in a concentration from about 0.01 pg/ml to about 100 mg/ml, characterized in that said composition is at a substantially neutral pH in a pharmaceutically acceptable form suitable for delivery to the eye to reduce ocular neovascularization and
(b) a steroid, an anti-prostoglandin, an inhibitor of metalloproteinase, a tetracycline, a macrolide antibiotic or any combination thereof.

14. The method of claim 13, wherein (b) is a steroid such as triamcinolone acetonide at a concentration from about 0.1 mg/ml to about 40 mg/ml and a tetracycline or a derivative thereof (including CMTs which inhibit MMP activity) such as doxycycline at a concentration from about 0.01 pg/ml to about 30 mg/ml or
wherein (b) is a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is an anti-prostaglandin such as flurbiprofen at a concentration from about 1 μg/ml to about 10 mg/ml and an macrolide antibiotic such as ascomycin at a concentration from about 20 μg/ml to about 200 μg/ml or
wherein (b) is an anti-prostaglandin such as flurbiprofen at a concentration from about 1 μg/ml to about 10 mg/ml and an inhibitor of a metalloproteinase in a concentration and dose to reduce ocular neovascularization or
wherein (b) is a steroid such as triamcinolone acetonide at a concentration from about 0.1 mg/ml to about 40 mg/ml and an inhibitor of a metalloproteinase in a concentration and dose to reduce ocular neovascularization or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an macrolide antibiotic such as ascomycin at a concentration from about 20 μg/ml to about 200 μg/ml or
wherein (b) is a tetracycline or a derivative thereof (including CMTs which inhibit MMP activity) such as doxycycline at a concentration from about 0.01 pg/ml to about 30 mg/ml and a steroid such as triamcinolone acetonide at a concentration from about 0.1 mg/ml to about 40 mg/ml and an anti-prostaglandin such as flurbiprofen at a concentration from about 1 μg/ml to about 10 mg/ml and an macrolide antibiotic such as ascomycin at a concentration from about 20 μg/ml to about 200 μg/ml.

15. The method of claim 11, wherein the method further comprises the step of administering an anti-angiogenic agent designed to block the actions of VEGF on endothelial cells or
wherein the method further comprises the step of administering an anti-angiogenic agent designed to block the actions of VEGF on endothelial cells and
wherein the anti-angiogenic agent is a rhuFab V2 or a humanized AMD-Fab or an anti-VEGF aptamer.
